(19) United States
(12) Patent Application Publication
(10) Pub. No.: US 2021/0163409 A1
(43) Pub. Date: Jun. 3, 2021

(54) ALTERNATE PROCESSES FOR THE PREPARATION OF PYRROLIDINE DERIVATIVES

(71) Applicant: DR. REDDY'S LABORATORIES LIMITED, Hyderabad (IN)

(72) Inventors: Srinivas ORUGANTI, Hyderabad (IN); Bhaskar KANDAGATLA, Hyderabad (IN); Saikat SEN, Kolkata (IN); Raju CHEERLAVANCHA, Hyderabad (IN); Magesh SAMPATH, Kancheepuram (IN); Martin FOX, Cambridge (GB); Vilas Hareshwar DAHANUKAR, Hyderabad (IN)

(21) Appl. No.: 16/630,257

(22) PCT Filed: Jul. 19, 2018
(86) PCT No.: PCT/IB2018/055368

§ 371 Date: Jan. 10, 2020

(30) Foreign Application Priority Data
Jul. 19, 2017 (IN) 201741025672

(51) Int. Cl. C07D 207/16

(52) U.S. Cl. CPC C07D 207/16

(57) ABSTRACT

Aspects of the present application relate to process for the preparation of Pyrrolidine derivatives useful as key intermediates for active ingredients. Specific aspects relate to alternate process for the preparation of Upadacitinib intermediate, 4-ethylpyrrolidine-3-carboxylic acid, its ester or a salt thereof. Processes disclosed here in are cost effective and industrially viable as compared to known processes.

Aspects of the present application relate to process for the preparation of a Pyrrolidine derivatives useful as key drug intermediates. Specific aspects of the present application relate to alternate process for the preparation of Upadacitinib intermediate, 4-ethylpyrrolidine-3-carboxylic acid, its ester or a salt thereof.

Upadacitinib is the adopted name of drug compound having a chemical name: (3S,4R)-3-ethyl-4-(3H-imidazo[1,2-a]pyrrolo[2,3-e]pyrazin-8-yl)-N-(2,2,2-trifluoroethyl)pyrrolidine-1-carboxamide and structure as below.

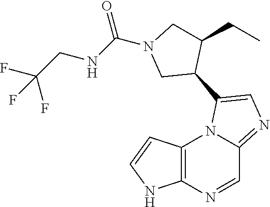 [SMILES: CC[C@@H]1CN(C(=O)NCC(F)(F)F)C[C@@H]1c1cnc2cnc3[nH]ccc3n12]

Upadacitinib, also known as ABT-494, is potent and selective JAK inhibitor under development for rheumatoid arthritis (RA). Upadacitinib is approximately 74 fold selective for Jak1 over Jak2 in cellular assays dependent on specific, relevant cytokines. Upadacitinib demonstrates efficacy in rat arthritis models. The researchers found that the proportion of patients meeting the American College of Rheumatology 20 percent improvement criteria (ACR20) at week 12 was higher with Upadacitinib than placebo (62 to 80 percent versus 46 percent). Patients were randomized to receive immediate-release Upadacitinib at 3, 6, 12, or 18 mg twice daily or matching placebo. The researchers found that significantly more patients receiving Upadacitinib achieved an ACR20 response (53 to 71 percent) versus placebo (34 percent). Preliminary evidence suggests that compared to Tofacitinib, Upadacitinib may spare Jak2 and Jak3 dependent signaling.

U.S. Pat. No. 6,245,801 B1 directed to alkyl pyrrolidine-3-carboxylic acids describes the preparation of trans 4-ethylpyrrolidine-3-carboxylic acid (3c) as per the route depicted below with about 78% yield.

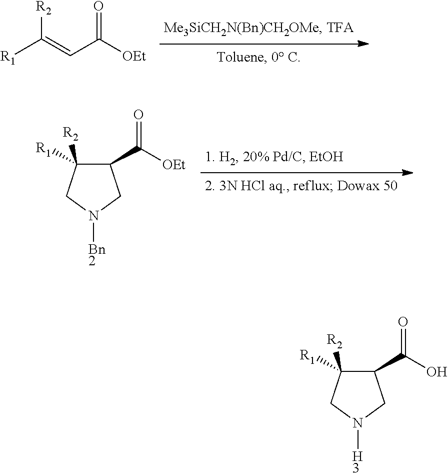

U.S. Pat. No. 8,426,411 B2 discloses Upadacitinib, its composition and use for treating diseases (incl. Rheumatoid arthritis). Therein, the synthetic preparation of Upadacitinib is not described specifically, however, a general route for the preparation of related compounds such as scheme II and for analogous compound [(3S,4R)-3-ethyl-4-(6H-pyrrolo[2,3-e][1,2,4]triazolo[4,3-a]pyrazin-1-yl)-N-(2,2,2-trifluoroethyl)pyrrolidine-1-carboxamide] is disclosed as example-36. U.S. Pat. No. 8,426,411 B2 discloses an approach as depicted in below scheme-A for the synthesis of Upadacitinib intermediate, 4-ethylpyrrolidine-3-carboxylic acid, its ester or a salt thereof.

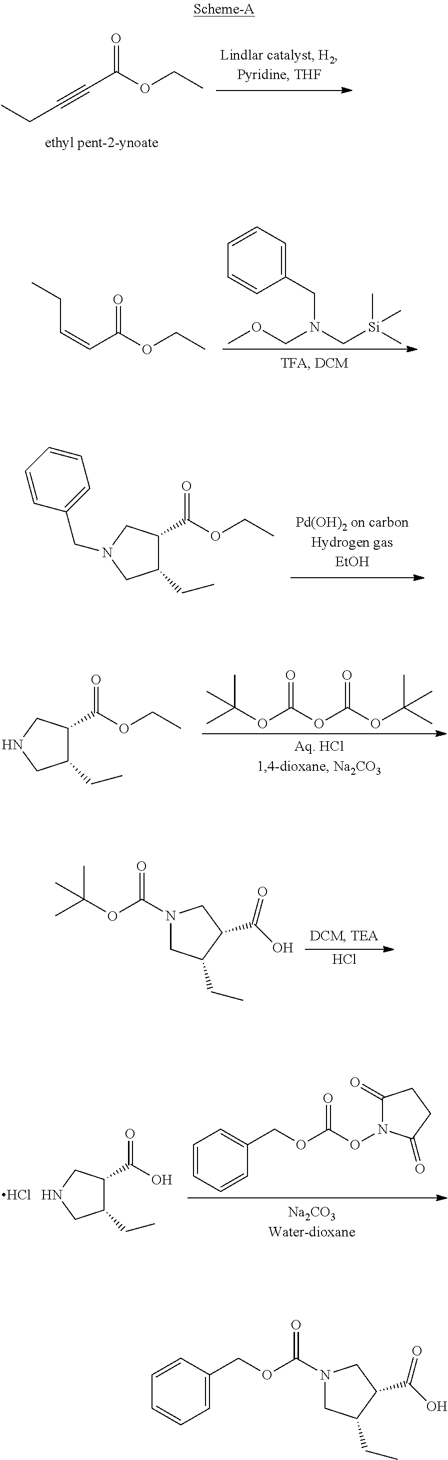

PCT application, WO 2015061665 A1 related to the method of treating Rheumatoid Arthritis and other autoimmune diseases with Upadacitinib, also discloses a similar route for the preparation of Upadacitinib as depicted below.

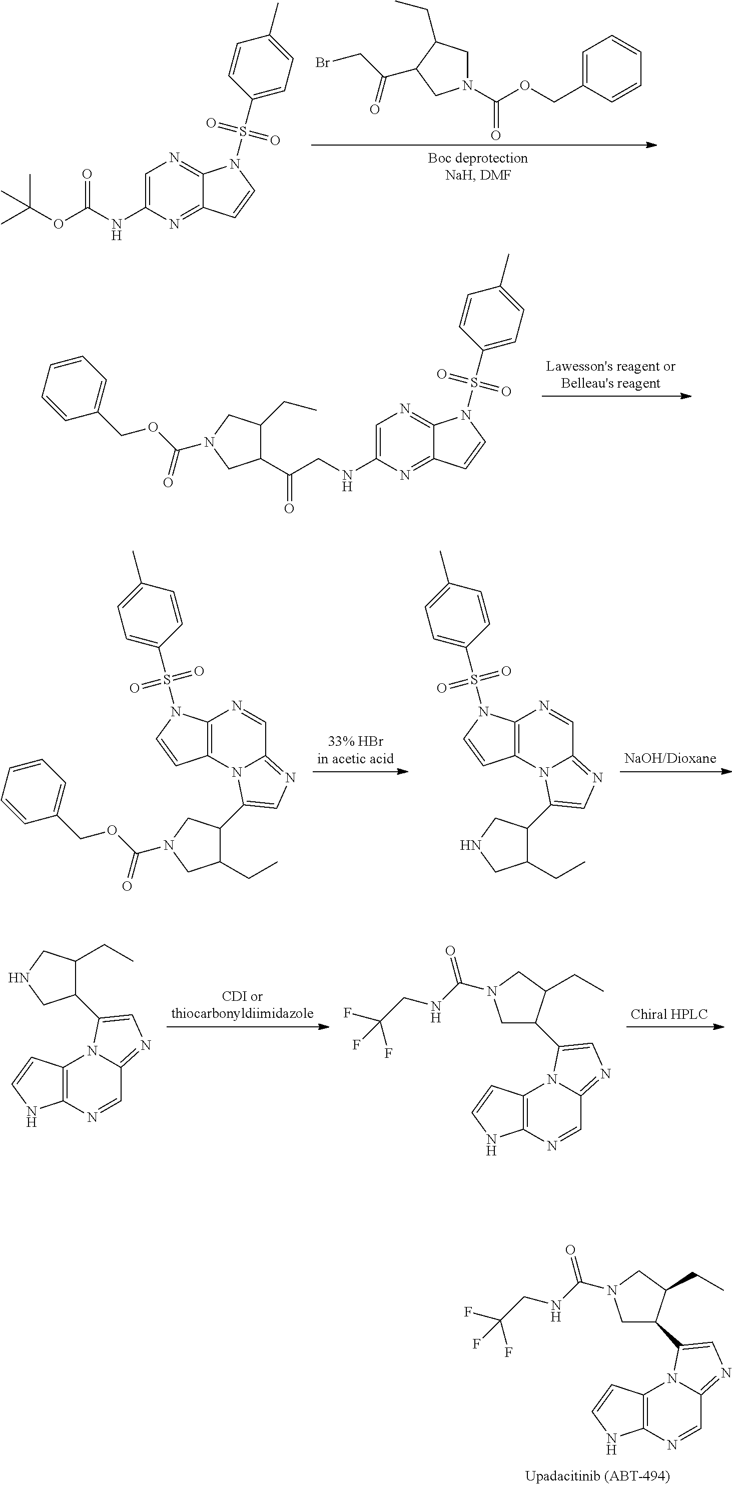

Prior art process may not be suitable at a large scale to obtain the product with desired quality and yield. Hence, there remains a need for alternate process for the preparation of 4-ethylpyrrolidine-3-carboxylic acid in a more cost effective and industrially viable manner.

### SUMMARY

In an aspect, the present application provides a process for the preparation of Upadacitinib intermediate, pyrrolidine-3-carboxylic acid, its ester or a salt thereof of formula (1), comprising the steps of
a) cyclizing 2-(1-nitrobutan-2-yl)malonate of formula (c) to 2-oxopyrrolidine-3-carboxylate of formula (d);

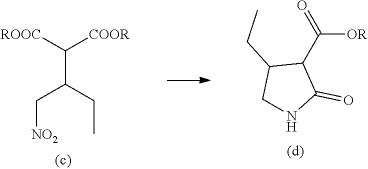

b) optionally, protecting 2-oxopyrrolidine-3-carboxylate of formula (d);

c) reducing the 2-oxopyrrolidine-3-carboxylate of formula (d) to pyrrolidine-3-carboxylic acid or an ester thereof of formula (1);

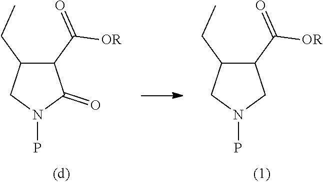

d) optionally, removing the protecting group of step b);

Wherein R may be hydrogen or group selected from the group comprising of alkyl, aryl, arylalkyl; and P is a hydrogen (or) nitrogen protecting group such as selected from the group comprising of tert-Butyloxycarbonyl (Boc), Benzyloxycarbonyl (Cbz), Benzyl (Bn).

In another aspect, the present application provides a process for the preparation of Upadacitinib intermediate, pyrrolidine-3-carboxylic acid, its derivative or a salt thereof of formula (1), comprising the steps of
a) reacting dialkyl malonate of formula (a) with Propionaldehyde to obtain dialkyl 2-propylidenemalonate of formula (b)

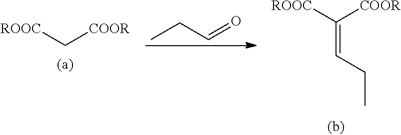

b) treating dialkyl 2-propylidenemalonate of formula (b) with nitromethane to obtain 2-(1-nitrobutan-2-yl) malonate of formula (c).

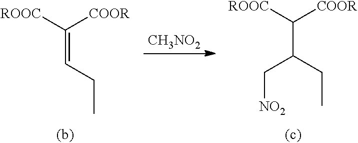

c) cyclizing 2-(1-nitrobutan-2-yl)malonate of formula (c) to 2-oxopyrrolidine-3-carboxylate of formula (d);

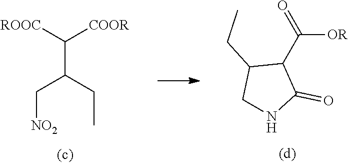

d) optionally, protecting 2-oxopyrrolidine-3-carboxylate of formula (d);

e) reducing the 2-oxopyrrolidine-3-carboxylate of formula (d) to pyrrolidine-3-carboxylate of formula (1);

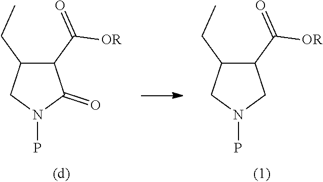

f) optionally, removing the protecting group of step d);

Wherein R may be hydrogen or group selected from the group comprising of alkyl, aryl, arylalkyl; and P is a hydrogen (or) nitrogen protecting group such as selected from the group comprising of tert-Butyloxycarbonyl (Boc), Benzyloxycarbonyl (Cbz), Benzyl (Bn).

Alternatively, the present application provides a process for the preparation of Upadacitinib intermediate, pyrrolidine-3-carboxylic acid, its derivative or a salt thereof of formula (1), comprising the step of cyclizing the alkyl pent-2-enoate,

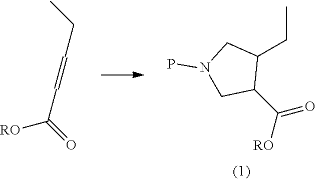

Wherein R may be hydrogen or group selected from the group comprising of alkyl, aryl, arylalkyl; and P is a nitrogen protecting group such as selected from the group comprising of tert-Butyloxycarbonyl (Boc), Benzyloxycarbonyl (Cbz), Benzyl (Bn).

In another aspect, the present application provides a process for the preparation of Upadacitinib intermediate, pyrrolidine-3-carboxylic acid, its ester or a salt thereof of formula (1), comprising the steps of
a) cyclizing the alkyl pent-2-ynoate to obtain alkyl 4-ethyl-2,5-dihydro-1H-pyrrole-3-carboxylate

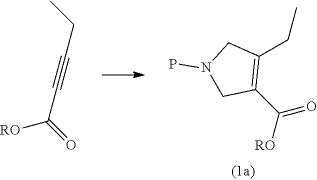

b) reducing the alkyl 4-ethyl-2,5-dihydro-1H-pyrrole-3-carboxylate of step a) to obtain compound of formula (1)

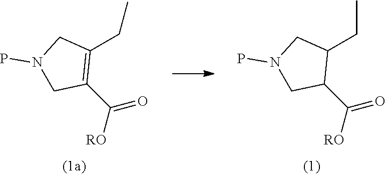

Wherein R may be hydrogen or group selected from the group comprising of alkyl, aryl, arylalkyl; and P is a nitrogen protecting group such as selected from the group comprising of tert-Butyloxycarbonyl (Boc), Benzyloxycarbonyl (Cbz), Benzyl (Bn).

In an aspect, the present application provides a process for the preparation of Upadacitinib intermediate, pyrrolidine-3-carboxylic acid, its derivative or a salt thereof of formula (1), comprising the steps of
a) reacting pent-2-ynoic acid or its derivative thereof with a chiral auxiliary such as an optically active sultam compound to obtain pent-2-ynamide

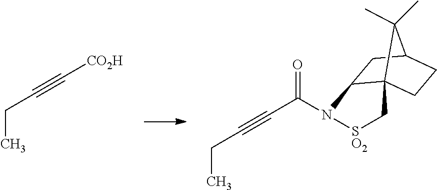

b) reducing the pent-2-ynamide of step a) to corresponding pent-2-enamide

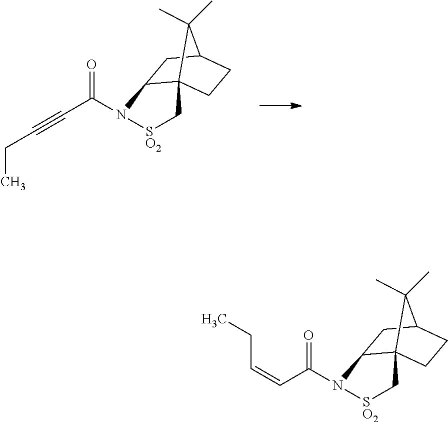 [SMILES: CCC#CC(=O)N1[C@H]2CC3CC[C@@]2(CS1(=O)=O)C3(C)C ; CC/C=C\C(=O)N1[C@H]2CC3CC[C@@]2(CS1(=O)=O)C3(C)C]

c) cyclizing the pent-2-enamide obtained in step b) to obtain pyrrolidine-3-carbamide, wherein P may be hydrogen or a nitrogen protecting group

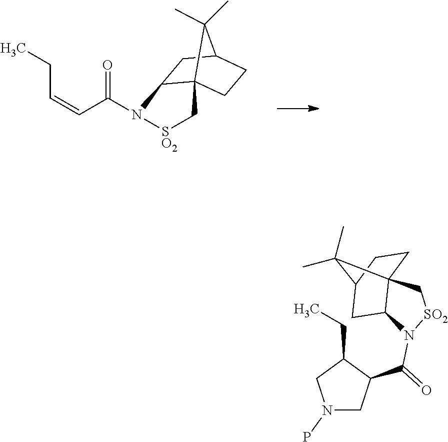

d) hydrolysis of pyrrolidine-3-carbamide of step c) to obtain pyrrolidine-3-carboxylic acid

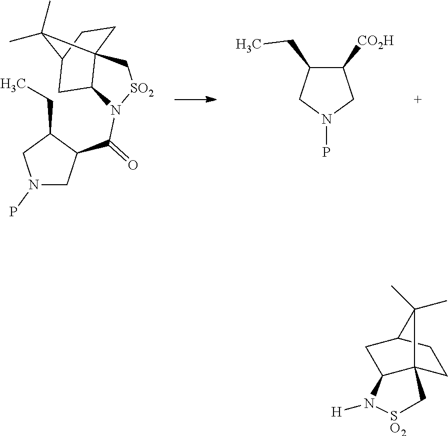

In an aspect, the present application provides a process for the preparation of Upadacitinib intermediate, pyrrolidine-3-carboxylic acid, its ester or a salt thereof of formula (1), comprising the steps of
a) cyclizing the pent-2-enamide to obtain pyrrolidine-3-carbamide, wherein P may be hydrogen or a nitrogen protecting group

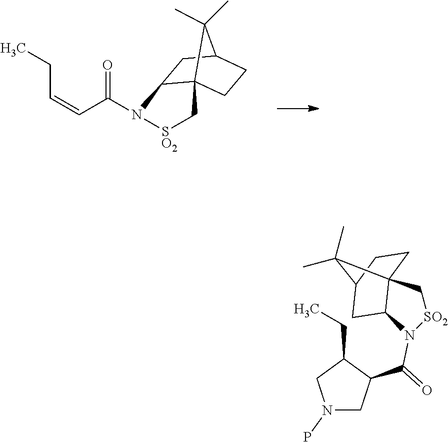

b) hydrolysis of pyrrolidine-3-carbamide of step c) to obtain pyrrolidine-3-carboxylic acid

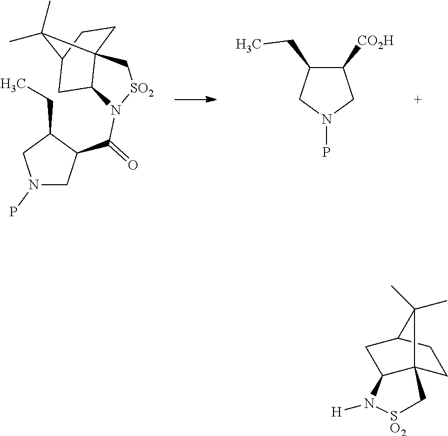

In another aspect, the present application provides process for the preparation of Upadacitinib or a salt thereof, comprising the steps of preparing of pyrrolidine-3-carboxylate of formula (1) according any of the aspects of the instant application and converting it to Upadacitinib or a salt thereof.

In another aspect, the present application provides process for the preparation of Upadacitinib or a salt thereof, comprising the steps of
a) resolving the pyrrolidine-3-carboxylic acid, its derivative or a salt thereof of formula (1) with optically active compound.
b) converting the optical isomer of pyrrolidine-3-carboxylic acid, its derivative or a salt thereof obtained in step a) to Upadacitinib or a salt thereof.

### DETAILED DESCRIPTION

In an aspect, the present application provides a process for the preparation of Upadacitinib intermediate, pyrrolidine-3-carboxylic acid, its ester or a salt thereof of formula (1), comprising the steps of
a) cyclizing 2-(1-nitrobutan-2-yl)malonate of formula (c) to 2-oxopyrrolidine-3-carboxylate of formula (d);

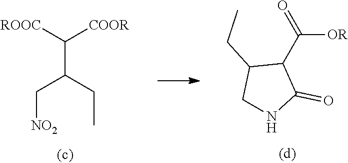

b) optionally, protecting 2-oxopyrrolidine-3-carboxylate of formula (d);
c) reducing the 2-oxopyrrolidine-3-carboxylate of formula (d) to pyrrolidine-3-carboxylic acid or an ester thereof of formula (1);

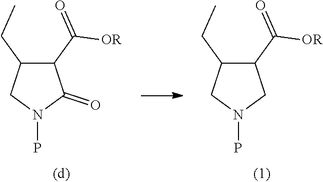

d) optionally, removing the protecting group of step b);

Wherein R may be hydrogen or group selected from the group comprising of alkyl, aryl, arylalkyl; and P is a hydrogen (or) nitrogen protecting group such as selected from the group comprising of tert-Butyloxycarbonyl (Boc), Benzyloxycarbonyl (Cbz), Benzyl (Bn).

In an embodiment, the staring material 2-(1-nitrobutan-2-yl)malonate of formula (c) may be purified either by recrystallization, chromatography, acid-base treatment or according to any other methods known in the art, before using.

In an embodiment, the staring material 2-(1-nitrobutan-2-yl)malonate of formula (c) may be prepared according to any of the methods described at any of the aspects and procedures exemplified in the instant specification or according to any other alternate methods known in the art.

In an embodiment, step a) of this aspect may be carried out by cyclizing 2-(1-nitrobutan-2-yl) malonate of formula (c) to 2-oxopyrrolidine-3-carboxylate of formula (d) through hydrogenation.

In an embodiment, the cyclization of 2-(1-nitrobutan-2-yl) malonate of formula (c) may be carried out by hydrogenating using molecular hydrogen such hydrogen gas in the presence of a suitable catalyst. Suitable catalyst includes, but not limited to palladium-on-carbon, platinum (IV) oxide, or Raney™ nickel, or the like. The cyclization of 2-(1-nitrobutan-2-yl) malonate of formula (c) may also be carried with under metal mediated reduction conditions such as zinc and acetic acid, zinc and hydrochloric acid, tin and hydrochloric acid, sodium amalgam in ethanol, or iron and acetic acid; tin chloride (II), titanium (III) chloride, or any other suitable reducing agent known in the art.

In an embodiment, the hydrogenation of 2-(1-nitrobutan-2-yl) malonate of formula (c) may be carried out in the presence of a suitable solvent. Suitable solvent includes, but not limited to alcohol solvent such as methanol, ethanol, isopropyl alcohol; ester solvent such as ethyl acetate, isopropyl acetate; ether solvent such tetrahydrofuran, 1,4-dioxane; hydrocarbons such as toluene; chlorinated solvent such as dichloromethane; water or mixtures thereof.

In an embodiment, cyclization reaction may be carried out at suitable temperature of about 0° C. to reflux temperature of the solvent used.

In an embodiment, the cyclization reaction may be carried out to complete the formation of 2-oxopyrrolidine-3-carboxylate of formula (d) for at least one hour or more.

Step b) of this aspect may be carried out by optionally protecting the Nitrogen of 2-oxopyrrolidine-3-carboxylate of formula (d) obtained at step a).

In embodiment, nitrogen protecting groups that may be employed at step b) may include, but not limited to 9-Fluorenylmethyl carbamate (Fmoc), Benzyl carbamate (Cbz), t-Butyl carbamate (Boc), Benzylamine (Bn), Phthalimide, Benzylideneamine, p-Toluenesulfonamide (Ts), Triphenylmethylamine (Tr), Acetamide, Trifluoroacetamide or the like.

In one embodiment, nitrogen protection of 2-oxopyrrolidine-3-carboxylate of formula (d) may be carried out by reacting it with protection source such as di-tert-butyl dicarbonate.

In an embodiment, nitrogen protection may be carried out in the presence of a suitable base such as metal hydroxides, metal carbonates or metal bicarbonates such as sodium hydroxide, sodium carbonate, sodium bicarbonate or the like. In alternate embodiment, the nitrogen protection may also be accomplished using an organic base such as triethyl amine, diisopropyl amine (DIPA), diisopropylethyl amine JDIPEA), 4-dimethylaminopyridine (DMAP) or mixture thereof.

In an embodiment, the nitrogen protection may be carried out in the presence of a suitable solvent. Suitable solvent includes, but not limited to ester solvent such as ethyl acetate, isopropyl acetate; ether solvent such tetrahydrofuran, 1,4-dioxane; hydrocarbons such as toluene, chlorinated hydrocarbon such as dichloromethane; alcohol solvent such as methanol, ethanol, isopropyl alcohol; or mixtures thereof.

In an embodiment, the nitrogen protection may be carried out at suitable temperature of about 0° C. to reflux temperature of the solvent used to complete the protection of nitrogen of 2-oxopyrrolidine-3-carboxylate of formula (d) for at least one hour or more.

Step c) of this aspect may be carried out by reducing the 2-oxopyrrolidine-3-carboxylate of formula (d) to pyrrolidine-3-carboxylic acid, its derivative or salt thereof of formula (1).

In an embodiment, reducing the 2-oxopyrrolidine-3-carboxylate of formula (d) may be carried out in direct single step without isolation of intermediate, 4,5-dihydro-1H-pyrrole-3-carboxylate of formula (d-i) in the presence of suitable reducing agent such as borane and the choice of reaction conditions should be made based on the tolerance of other functional groups to the acidic or basic conditions.

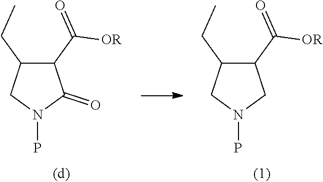

In an alternate embodiment, reduction of 2-oxopyrrolidine-3-carboxylate of formula (d) may be carried out in following steps

(i) Through the formation 4,5-dihydro-1H-pyrrole-3-carboxylate of formula (d-i); and

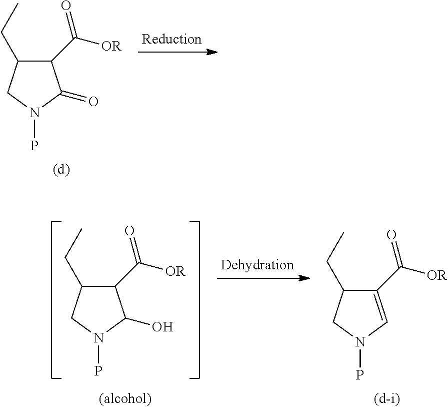

(ii) Hydrogenation of 4,5-dihydro-1H-pyrrole-3-carboxylate

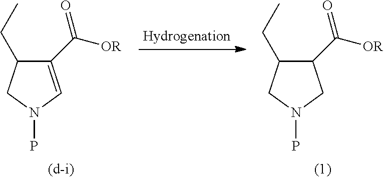

In an embodiment, 2-oxopyrrolidine-3-carboxylate of formula (d) may be treated with a suitable reducing agent to obtain corresponding alcohol, which on subsequent dehydration results in 4,5-dihydro-1H-pyrrole-3-carboxylate of formula (d-i).

Suitable reducing agent that can reduce the carbonyl group to corresponding alcohol group may be selected from the group comprising of hydrides of boron and Aluminium or derivatives thereof such as sodium borohydride, potassium borohydride, Lithium borohydride, sodium cyano borohydride, lithium alkyl borohydride, sodium dihydro-bis-(2-methoxyethoxy) aluminate solution (VITRIDE®), diisobutyl aluminium hydride, or the like.

In an embodiment, reduction of 2-oxopyrrolidine-3-carboxylate of formula (d) may be carried out at a suitable temperature of about −70° C. to 0° C. to complete the reduction of oxo-group to alcohol for at least 1 hour or more.

In an embodiment, reduction may be carried out in the presence of a suitable solvent. Suitable solvent includes, but not limited to ester solvent such as ethyl acetate, isopropyl acetate; ether solvent such as tetrahydrofuran, 1,4-dioxane; hydrocarbons such as, toluene; chlorinated hydrocarbon such as dichloromethane; alcohol solvent such as methanol, ethanol, isopropyl alcohol; or mixtures thereof.

In an embodiment, the alcohol obtained from the reduction of 2-oxopyrrolidine-3-carboxylate of formula (d) may be isolated and/or purified or directly taken for the dehydration step, without isolation and purification.

Dehydration of alcohol may be carried out by treating the alcohol with strong acid catalyst to obtain 4,5-dihydro-1H-pyrrole-3-carboxylate of formula (d-i). Acid catalyst may include but not limited to sulphuric acid, phosphoric acid or other acid derivatives thereof. In an embodiment, dehydration of alcohol may be carried out by treating the alcohol with a strong acid cation exchange resin such as TULSION T-66 MP, Indion® 190, SK104H, SK110, SK112, SK1B or the like.

In an embodiment, dehydration of alcohol may be carried out at a suitable temperature of about 0° C. to reflux temperature of the solvent used to complete the formation of 4,5-dihydro-1H-pyrrole-3-carboxylate of formula (d-i) for at least 1 hour or more.

In an embodiment, dehydration may be carried out in the presence of a suitable solvent. Suitable solvent includes, but not limited to ester solvent such as ethyl acetate, isopropyl acetate; ether solvent such tetrahydrofuran, 1,4-dioxane; hydrocarbons such as toluene; chlorinated hydrocarbon such as dichloromethane; alcohol solvent such as methanol, ethanol, isopropyl alcohol; or mixtures thereof.

In an embodiment, the hydrogenation of 4,5-dihydro-1H-pyrrole-3-carboxylate of formula (d-i) to obtain pyrrolidine-3-carboxylic acid, its ester or salt thereof of formula (1) may be carried out by treating with hydrogen in the presence of a suitable catalyst. Suitable catalyst includes, but not limited to catalytic hydrogenation using palladium-on-carbon, platinum (IV) oxide, or Raney™ nickel, or the like. The cyclization of 2-(1-nitrobutan-2-yl) malonate of formula (c) may also be carried with under metal mediated reduction conditions such as zinc and acetic acid, zinc and hydrochloric acid, tin and hydrochloric acid, sodium amalgam in ethanol, or iron and acetic acid; tin chloride (II), titanium (III) chloride, or any other suitable reducing agent known in the art.

In an embodiment, hydrogenation may be carried out in the presence of a suitable solvent. Suitable solvent includes, but not limited to alcohol solvent such as methanol, ethanol, isopropyl alcohol; ester solvent such as ethyl acetate, isopropyl acetate; ether solvent such tetrahydrofuran, 1,4-dioxane; hydrocarbons such as toluene; chlorinated hydrocarbon such as dichloromethane; or mixtures thereof.

In an embodiment, hydrogenation may be carried out at a suitable temperature of about 0° C. to reflux temperature of the solvent used to complete the formation of to obtain pyrrolidine-3-carboxylic acid, its ester or salt thereof of formula (1) for at least 1 hour or more.

Step d) of this aspect may be carried out by removing the protecting group to obtain pyrrolidine-3-carboxylic acid, its ester or salt thereof of formula (1).

In embodiment, the protecting group on nitrogen of pyrrolidine-3-carboxylic acid, its ester or salt thereof of formula (1) may be removed employing suitable methods known in the art or procedures described or exemplified in the instant application.

In an embodiment, the nitrogen protecting group may be removed by treating the N-protected pyrrolidine-3-carboxylic acid, its ester or salt thereof of formula (1) with suitable acids such as hydrochloric acid or trifluoroacetic acid (TFA), preferably in diluted form or any alternate conditions known in the art, Based on the protecting group used, any other alternate conditions may be employed, for example, may be carried out through hydrogenation in the presence of palladium catalyst such as palladium on carbon, palladium hydroxide, or the like, optionally in the presence of an acid.

In an embodiment, removal of nitrogen protecting group may be carried out in the presence of a suitable solvent selected from the group comprising of ester solvent such as ethyl acetate, isopropyl acetate; ether solvent such tetrahydrofuran, 1,4-dioxane; hydrocarbons such as toluene; chlorinated hydrocarbon such as dichloromethane; alcohol solvent such as methanol, ethanol, isopropyl alcohol; or mixtures thereof.

In an embodiment, removal of nitrogen protecting group may be carried out at suitable temperature of about 0° C. to reflux temperature of the solvent used to completely remove the protecting group for at least one hour or more.

In an embodiment, the pyrrolidine-3-carboxylic acid, its ester or salt thereof of formula (1) obtained by the process of this aspect may be purified by suitable method such as recrystallization, chromatography, acid-base treatment or alternate purification methods known in the art.

In an embodiment, the pyrrolidine-3-carboxylic acid, its ester or salt thereof of formula (1) obtained by the process of this aspect may be converted to Upadacitinib according to any of the procedure described or exemplified in the instant application.

In another aspect, the present application provides a process for the preparation of Upadacitinib intermediate, pyrrolidine-3-carboxylic acid, its ester or a salt thereof of formula (1), comprising the steps of
a) reacting dialkyl malonate of formula (a) with Propionaldehyde to obtain dialkyl 2-propylidenemalonate of formula (b)

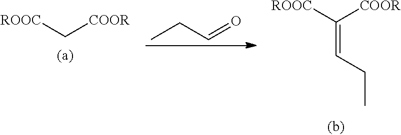

b) treating dialkyl 2-propylidenemalonate of formula (b) with nitromethane to obtain 2-(1-nitrobutan-2-yl) malonate of formula (c).

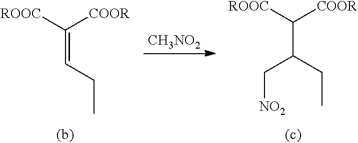

c) cyclizing 2-(1-nitrobutan-2-yl)malonate of formula (c) to 2-oxopyrrolidine-3-carboxylate of formula (d);

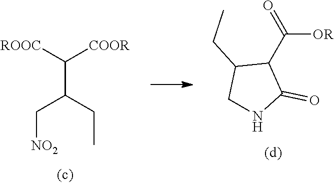

d) optionally, protecting 2-oxopyrrolidine-3-carboxylate of formula (d);
e) reducing the 2-oxopyrrolidine-3-carboxylate of formula (d) to pyrrolidine-3-carboxylate of formula (1);

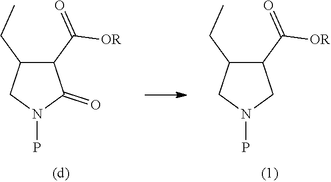

f) optionally, removing the protecting group of step b);

Wherein R may be hydrogen or group selected from the group comprising of alkyl, aryl, arylalkyl; and P is a hydrogen (or) nitrogen protecting group such as selected from the group comprising of tert-Butyloxycarbonyl (Boc), Benzyloxycarbonyl (Cbz), Benzyl (Bn).

Dialkyl malonate of formula (a) of step a) may be commercially available or may be prepared and purified according to methods known in the art.

Step a) of this aspect may be carried out treating dialkyl malonate of formula (a) with Propionaldehyde in the presence of a suitable base to obtain dialkyl 2-propylidenemalonate of formula (b).

Suitable base may be selected from the group comprising of an inorganic or organic base such as pyridine, piperidine, tert-butoxides like potassium tert-butoxide, organolithium reagents like n-BuLi, PhLi, MeLi, lithium diisopropamide (LDA); or the like.

Optionally the reaction between dialkyl malonate of formula (a) and Propionaldehyde may be carried out with an additional presence of an acid such as acetic acid.

In an embodiment, the reaction between dialkyl malonate of formula (a) and Propionaldehyde may be carried out in the presence of a suitable solvent selected from the group comprising of ester solvent such as ethyl acetate, isopropyl acetate; ether solvent such tetrahydrofuran, 1,4-dioxane; hydrocarbons such as toluene; chlorinated hydrocarbon such as dichloromethane; or mixtures thereof.

In an embodiment, the reaction between dialkyl malonate of formula (a) and Propionaldehyde may be carried out at suitable temperature of about 0° C. to reflux temperature of the solvent used to complete the formation of dialkyl 2-propylidenemalonate of formula (b) for at least one hour or more.

Step b) of this aspect may be carried out by treating dialkyl 2-propylidenemalonate of formula (b) with nitromethane to obtain 2-(1-nitrobutan-2-yl) malonate of formula (c). In embodiment, nitromethane may be added to the propylidenemalonate under condition suitable to obtain 2-(1-nitrobutan-2-yl) malonate.

In an embodiment, dialkyl 2-propylidenemalonate of formula (b) may be treated with nitromethane in the presence of a suitable catalyst. Suitable catalyst includes, but not limited to 1,8-diazabicyclo[5.4.0]undec-7-ene [DBU], 1,4-diazabicyclo[2.2.2]octane (DABCO), 1,5-Diazabicyclo[4.3.0]non-5-ene (DBN) or the like.

In an embodiment, dialkyl 2-propylidenemalonate of formula (b) may be treated with nitromethane in the presence of a suitable solvent. Suitable solvent includes, but not limited to ester solvent such as ethyl acetate, isopropyl acetate; ether solvent such tetrahydrofuran, 1,4-dioxane; hydrocarbons such as dichloromethane, toluene; or mixtures thereof.

In an embodiment, dialkyl 2-propylidenemalonate of formula (b) may be treated with nitromethane at suitable temperature of about 0° C. to reflux temperature of the solvent used to complete the formation of 2-(1-nitrobutan-2-yl) malonate of formula (c) for at least one hour or more.

Steps c) to 0 of the instant aspect may be carried as per the steps a) to d) of the previous aspect for the preparation of pyrrolidine-3-carboxylate of formula (1) or according to the procedures described or exemplified in the instant application.

In another aspect, the present application provides a process for the preparation of Upadacitinib intermediate, pyrrolidine-3-carboxylic acid, its ester or a salt thereof of formula (1), comprising the step of cyclizing the alkyl pent-2-enoate,

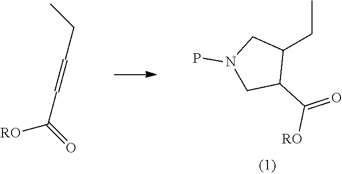

Wherein R may be hydrogen or group selected from the group comprising of alkyl, aryl, arylalkyl; and P is a nitrogen protecting group such as selected from the group comprising of tert-Butyloxycarbonyl (Boc), Benzyloxycarbonyl (Cbz), Benzyl (Bn).

In embodiments, the starting material, alkyl pent-2-enoate may be prepared according to the methods known in the art or according to the procedures described or exemplified in the instant application.

In an embodiment, the alkyl pent-2-enoate may be obtained by the hydrogenation of alkyl pent-2-ynoate in the presence of suitable catalyst.

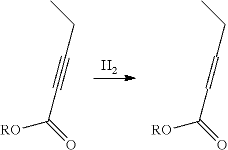

Suitable catalysts include Lindlar catalyst, Raney™ nickel or any other suitable catalyst known in the art.

In embodiments, the cyclization of alkyl pent-2-enoate may be carried out by treating it with suitable amine compound. Suitable amine compound includes but not limited to N-protected-1-methoxy-N-alkylsilyl methylmethaneamine such as N-benzyl-1-methoxy-N-trimethylsilyl methylmethaneamine.

In embodiments, the cyclization of alkyl pent-2-enoate may be carried out by in the presence of a suitable catalyst. Suitable catalyst includes, but not limited to Trifluoroacetic acid (TFA).

In embodiments, the cyclization may be carried out in the presence of a suitable solvent. Suitable solvent includes, but not limited to hydrocarbons such as dichloromethane, toluene; ester solvent such as ethyl acetate, isopropyl acetate; ether solvent such tetrahydrofuran, 1,4-dioxane or the mixtures thereof.

In embodiments, the cyclization may be carried out at suitable temperature of about −30° C. to reflux temperature of the solvent used to complete the cyclization of alkyl pent-2-enoate for at least one hour or more.

In another aspect, the present application provides a process for the preparation of Upadacitinib intermediate, pyrrolidine-3-carboxylic acid, its ester or a salt thereof of formula (1), comprising the steps of
a) cyclizing the alkyl pent-2-ynoate to obtain alkyl 4-ethyl-2,5-dihydro-1H-pyrrole-3-carboxylate

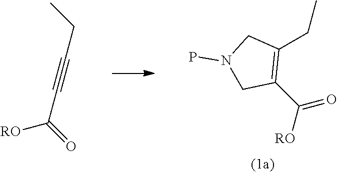

b) reducing the alkyl 4-ethyl-2,5-dihydro-1H-pyrrole-3-carboxylate of step a) to obtain compound of formula (1)

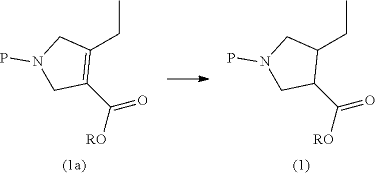

Wherein R may be hydrogen or group selected from the group comprising of alkyl, aryl, arylalkyl; and P is a nitrogen protecting group such as selected from the group comprising of tert-Butyloxycarbonyl (Boc), Benzyloxycarbonyl (Cbz), Benzyl (Bn).

Step a) of this aspect may be carried out through the cyclization of alkyl pent-2-ynoate may be carried out by treating it with suitable amine compound. Suitable amine compound includes but not limited to N-protected-1-methoxy-N-alkylsilyl methylmethaneamine such as N-benzyl-1-methoxy-N-trimethylsilyl methylmethaneamine.

In embodiments, the cyclization of alkyl pent-2-ynoate may be carried out by in the presence of a suitable catalyst. Suitable catalyst includes, but not limited to Trifluoroacetic acid (TFA).

In embodiments, the cyclization may be carried out in the presence of a suitable solvent. Suitable solvent includes, but not limited to hydrocarbons such as dichloromethane, toluene; ester solvent such as ethyl acetate, isopropyl acetate; ether solvent such tetrahydrofuran, 1,4-dioxane or the mixtures thereof.

In embodiments, the cyclization may be carried out at suitable temperature of about −30° C. to reflux temperature of the solvent used to complete the cyclization of alkyl pent-2-ynoate for at least one hour or more.

Step b) of this aspect may be carried out by the reduction of alkyl 4-ethyl-2,5-dihydro-1H-pyrrole-3-carboxylate of step a).

In an embodiment, the reduction of alkyl 4-ethyl-2,5-dihydro-1H-pyrrole-3-carboxylate to obtain pyrrolidine-3-carboxylic acid, its ester or salt thereof of formula (1) may be carried out by treating with hydrogen in the presence of a suitable catalyst. Suitable catalyst includes, but not limited to catalytic hydrogenation using palladium-on-carbon, platinum (IV) oxide, or Raney™ nickel, or any other suitable reducing agent known in the art.

In an embodiment, reduction may be carried out in the presence of a suitable solvent. Suitable solvent includes, but not limited to alcohol solvent such as methanol, ethanol, isopropyl alcohol; ester solvent such as ethyl acetate, isopropyl acetate; ether solvent such tetrahydrofuran, 1,4-dioxane; chlorinated hydrocarbons such as dichloromethane, toluene; or mixtures thereof.

In an embodiment, reduction be carried out at a suitable temperature of about 0° C. to reflux temperature of the solvent used to complete the formation of to obtain pyrrolidine-3-carboxylic acid, its ester or salt thereof of formula (1) for at least 1 hour or more.

In an aspect, the present application provides process for the preparation of Upadacitinib or a salt thereof, comprising the steps of preparing of pyrrolidine-3-carboxylate of formula (1) according any of the aspects of the instant application and converting it to Upadacitinib or a salt thereof.

In an aspect, the present application provides a process for the preparation of Upadacitinib intermediate, pyrrolidine-3-carboxylic acid, its ester or a salt thereof of formula (1), comprising the steps of
a) reacting pent-2-ynoic acid or its derivative thereof with a chiral auxiliary such as an optically active sultam compound to obtain pent-2-ynamide

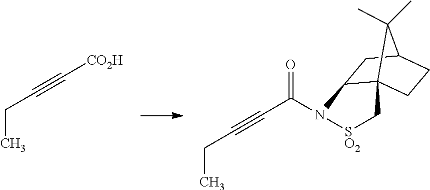

b) reducing the pent-2-ynamide of step a) to corresponding pent-2-enamide

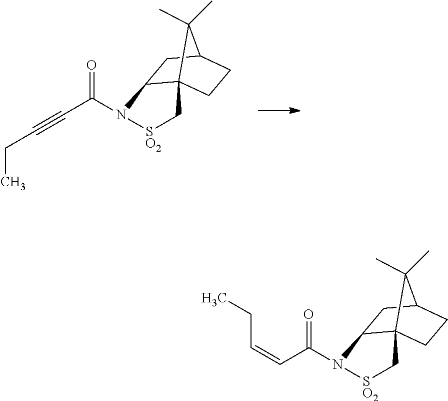 [SMILES: CCC#CC(=O)N1[C@H]2CC3CC[C@@]2(CS1(=O)=O)C3(C)C ; CC/C=C\C(=O)N1[C@H]2CC3CC[C@@]2(CS1(=O)=O)C3(C)C]

c) cyclizing the pent-2-enamide obtained in step b) to obtain pyrrolidine-3-carbamide, wherein P may be hydrogen or a nitrogen protecting group

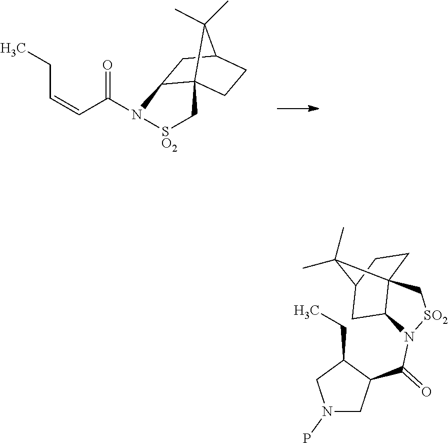

d) hydrolysis of pyrrolidine-3-carbamide of step c) to obtain pyrrolidine-3-carboxylic acid

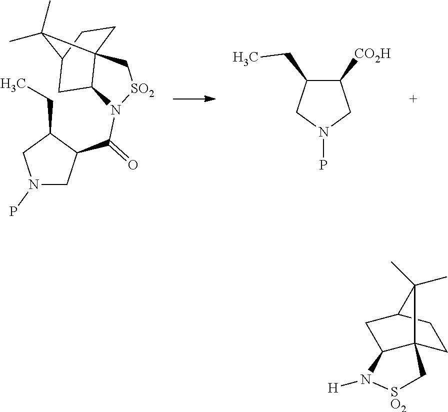

The starting material, pent-2-ynoic acid or its derivative may be obtained according any of the methods reported in the literature or according to the procedures described or exemplified in the instant application. In an embodiment, the pent-2-ynoic acid or its derivative may be obtained by the hydrolysis of corresponding ester as depicted below, wherein R is any alky, arylalkyl or aryl group.

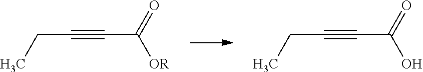

Step a) of this aspect may be carried out by the reaction of pent-2-ynoic acid or its derivative with a chiral auxiliary such as an optically active sultam compound. Optically active sultam compound includes, but not limited to (1R)-(+)-2,10-Camphorsultam, (1S)-(−)-2,10-Camphorsultam or the like.

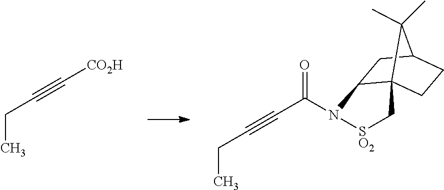

In an embodiment, the pent-2-ynoic acid may be converted to its reactive derivative such as a mixed anhydride with pivaloyl chloride before reacting it with optically active sultam compound. In embodiments, the reactive derivative may be formed in situ without isolating prior to reacting with sultam compound.

The mixed anhydride formation with pivaloyl chloride may be carried out in the presence of a suitable base that includes, but not limited to an organic amine selected from the group comprising of triethylamine, diisopropylamine, diisopropylethylamine or the like.

The reaction between pent-2-ynoic acid or its derivative with an optically active sultam compound may be carried out in the presence of a suitable base, selected from the group comprising of methyllithium, n-butyllithium, phenyllithium, lithium diisopropylamide or the like.

The reaction between pent-2-ynoic acid or its derivative with an optically active sultam compound may be carried out in the presence of an suitable solvent selected from the group comprising of ether solvent such tetrahydrofuran, 1,4-dioxane; ester solvent such as ethyl acetate, isopropyl acetate; chlorinated hydrocarbons such as dichloromethane, toluene; or mixtures thereof.

The reaction between pent-2-ynoic acid or its derivative with an optically active sultam compound may be carried out at suitable temperature of about −100 to 0° C. to complete the formation of pent-2-ynamide for at least one hour or more.

Step b) of this aspect may be carried out by reducing the pent-2-ynamide of step a) under suitable conditions.

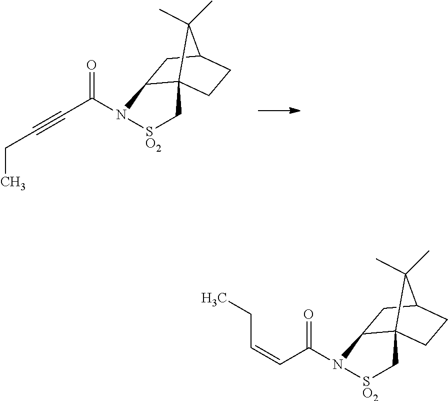 [SMILES: CCC#CC(=O)N1[C@H]2CC3CC[C@@]2(CS1(=O)=O)C3(C)C ; CC/C=C\C(=O)N1[C@H]2CC3CC[C@@]2(CS1(=O)=O)C3(C)C]

In an embodiment, the reduction of pent-2-ynamide to obtain corresponding pent-2-enamide may be carried out by treating with hydrogen in the presence of a suitable catalyst.

Suitable catalysts include Lindlar catalyst, Raney™ nickel or any other suitable catalyst known in the art.

In an embodiment, reduction may be carried out in the presence of an inert solvent. Inert solvent includes, but not limited to alcohol solvent such as methanol, ethanol, isopropyl alcohol; ester solvent such as ethyl acetate, isopropyl acetate; ether solvent such tetrahydrofuran, 1,4-dioxane; chlorinated hydrocarbons such as dichloromethane toluene; or mixtures thereof.

In an embodiment, reduction may be carried out at a suitable temperature of about 0° C. to reflux temperature of the solvent used to complete the formation of to obtain pent-2-enamide for at least 1 hour or more.

Step c) may be carried out by cyclizing the pent-2-enamide obtained in step b) to obtain pyrrolidine-3-carbamide, wherein P may be hydrogen or a nitrogen protecting group.

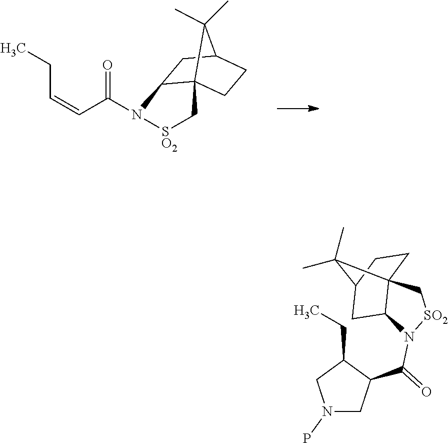

Cyclization of the pent-2-enamide may be carried out by treating it with suitable amine compound. Suitable amine compound includes but not limited to N-protected-1-methoxy-N-alkylsilyl methylmethaneamine such as N-benzyl-1-methoxy-N-trimethylsilyl methylmethaneamine.

In embodiments, the cyclization of pent-2-enamide may be carried out by in the presence of a suitable catalyst. Suitable catalyst includes, but not limited to Trifluoroacetic acid (TFA).

Cyclization may be carried out in the presence of a suitable solvent. Inert solvent includes, but not limited to chlorinated hydrocarbons such as dichloromethane, toluene; ester solvent such as ethyl acetate, isopropyl acetate; ether solvent such tetrahydrofuran, 1,4-dioxane or the mixtures thereof.

Cyclization may be carried out at suitable temperature of about −30° C. to reflux temperature of the solvent used to complete the cyclization of pent-2-enamide for at least one hour or more.

Step d) of this aspect may be carried by the hydrolysis of pyrrolidine-3-carbamide obtained in step c) to obtain pyrrolidine-3-carboxylic acid.

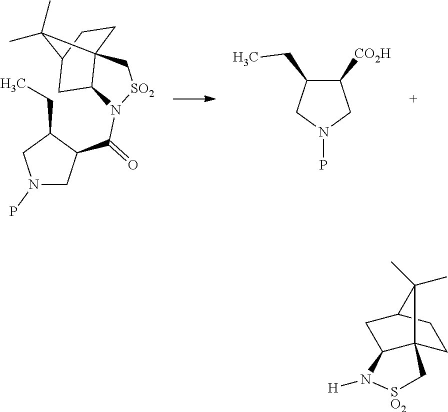

Hydrolysis of pyrrolidine-3-carbamide may be carried out in the presence of an acid or a base. Acid may include, but not limited to hydrogen halides such as HCl, HBr, HI; sulphuric acid, phosphoric acid, nitric acid or the like and a base may include, but not limited to hydroxides such as NaOH, LiOH, KOH, Mg(OH)₂, Ca(OH)₂ or the like; alkoxides such as methoxides, ethoxides, t-butoxides or the like; carbonates such as sodium carbonate, potassium carbonate and the like

Hydrolysis of pyrrolidine-3-carbamide may be carried out in the presence of a suitable solvent which include, but not limited to ethers such as tetrahydrofuran, 1,4-dioxane or the like; alcohols such as tert-butanol or the like; ketones such as methyl tert-butyl ketone or the like and hydrocarbons such as toluene or the like.

Hydrolysis may be carried out at suitable temperature of about −30° C. to reflux temperature of the solvent used to complete the formation of pyrrolidine-3-carboxylic acid for at least one hour or more.

In an aspect, the present application provides a process for the preparation of Upadacitinib intermediate, pyrrolidine-3-carboxylic acid, its ester or a salt thereof of formula (1), comprising the steps of
a) cyclizing the pent-2-enamide to obtain pyrrolidine-3-carbamide

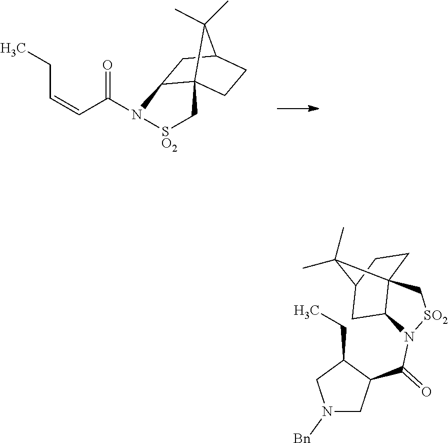

b) hydrolysis of pyrrolidine-3-carbamide of step c) to obtain pyrrolidine-3-carboxylic acid

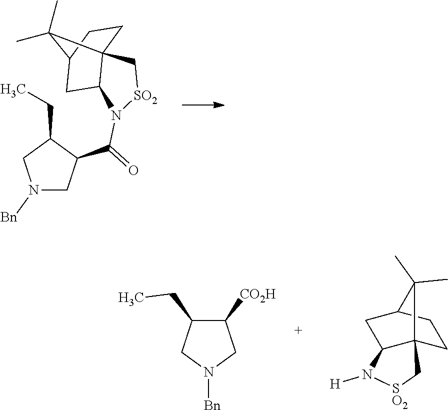 [SMILES: CC[C@@H]1CN(Cc2ccccc2)C[C@@H]1C(=O)N1[C@H]2CC3CC[C@@]2(CS1(=O)=O)C3(C)C ; CC[C@@H]1CN(Cc2ccccc2)C[C@@H]1C(=O)O ; CC1(C)C2CC[C@@]13CS(=O)(=O)N[C@H]3C2]

In embodiments, the starting material may be obtained through either of the following methodologies.

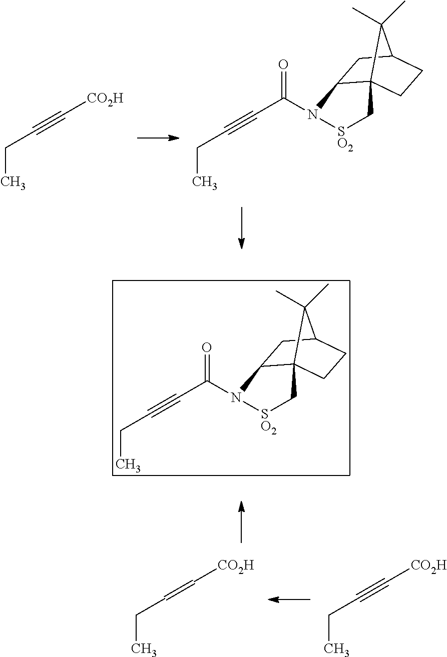

In embodiments, steps a) and b) may be carried out according to the methods known in the art or according to the procedures described in any of the previous aspects or exemplified in the instant application.

In an aspect, the present application provides process for the preparation of Upadacitinib or a salt thereof, comprising the steps of
a) resolving the pyrrolidine-3-carboxylic acid, its derivative or a salt thereof of formula (1) with an optically active compound.
b) converting the optical isomer of pyrrolidine-3-carboxylic acid, its derivative or a salt thereof obtained in step a) to Upadacitinib or a salt thereof.

In embodiments, the pyrrolidine-3-carboxylic acid, its ester or a salt thereof of formula (1) may be prepared according any method known in the art or according to procedures described or exemplified in the instant application.

Step a) of this aspect may be carried out by treating the pyrrolidine-3-carboxylic acid, its ester or a salt thereof of formula (1) with an optically active compound.

In embodiments, the optically active compound may be selected from the group comprising of an optically active amine such as R- and/or S-1-Naphthyl ethyl amine, R- and/or S-1-phenyl ethyl amine, or the like.

In embodiments, resolution of pyrrolidine-3-carboxylic acid of formula (1) may be carried out by treating it with R- and/or S-1-Naphthyl ethyl amine.

In an embodiment, the pyrrolidine-3-carboxylic acid of formula (1) may be treated with R- and/or S-1-Naphthyl ethyl amine.

In an embodiment, the pyrrolidine-3-carboxylic acid of formula (1) may be treated with S-1-Naphthyl ethyl amine to obtain salt of (3S,4R)-pyrrolidine-3-carboxylic acid of formula (1) with S-1-Naphthyl ethyl amine and (3R,4S)-pyrrolidine-3-carboxylic acid of formula (1) in free form as depicted below.

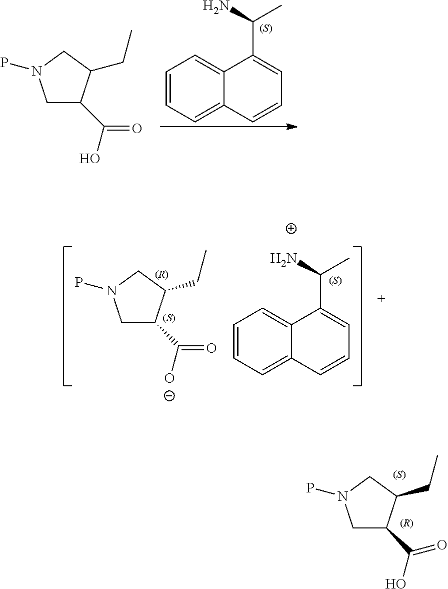

In embodiments, the precipitated salt of (3S,4R)-pyrrolidine-3-carboxylic acid of formula (1) with S-1-Naphthyl ethyl amine may be separated from (3R,4S)-pyrrolidine-3-carboxylic acid of formula (1) in free form as mother liquor.

In further embodiment, the obtained mother liquor containing free form of (3R,4S)-pyrrolidine-3-carboxylic acid of formula (1) may be treated with R-1-Naphthyl ethyl amine to obtain the salt of (3R,4S)-pyrrolidine-3-carboxylic acid of formula (1) with R-1-Naphthyl ethyl amine.

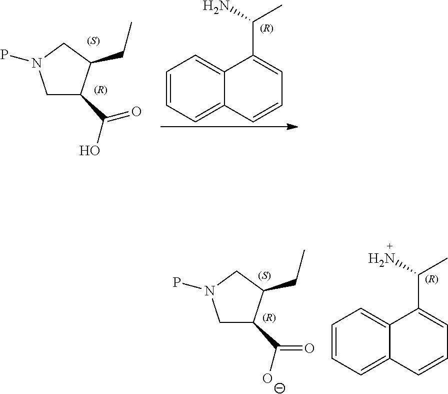

In embodiments, salt of (3R,4S)-pyrrolidine-3-carboxylic acid of formula (1) with R-1-Naphthyl ethyl amine may be treated with a suitable acid to obtain the free form of (3R,4S)-pyrrolidine-3-carboxylic acid of formula (1)

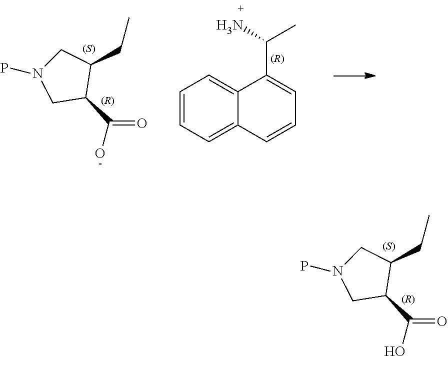

Suitable acid may include, but not limited to inorganic acid such as hydrochloric acid, hydrobromic acid, sulfuric acid, phosphoric acid, ammonium chloride; organic acid such as acetic acid, methane sulfonic acid, formic acid; or the like. In an embodiment, the acid may be used in diluted or concentrated form.

Step b) of this aspect involves the conversion of optical isomer of pyrrolidine-3-carboxylic acid, its ester or a salt thereof obtained in step a) to Upadacitinib or a salt thereof.

In embodiments, (3R,4S)-pyrrolidine-3-carboxylic acid, its ester or a salt thereof of formula (1) may be converted to Upadacitinib or a salt thereof according to methods known in the art or according to procedures described or exemplified in the instant application.

In an embodiment, the (3R,4S)-pyrrolidine-3-carboxylic acid, its ester or a salt thereof of formula (1) may be converted to (3R,4S)-3-(2-haloacetyl)-4-ethylpyrrolidine-1-carboxylate and then to Upadacitinib or intermediates thereof, as depicted below.

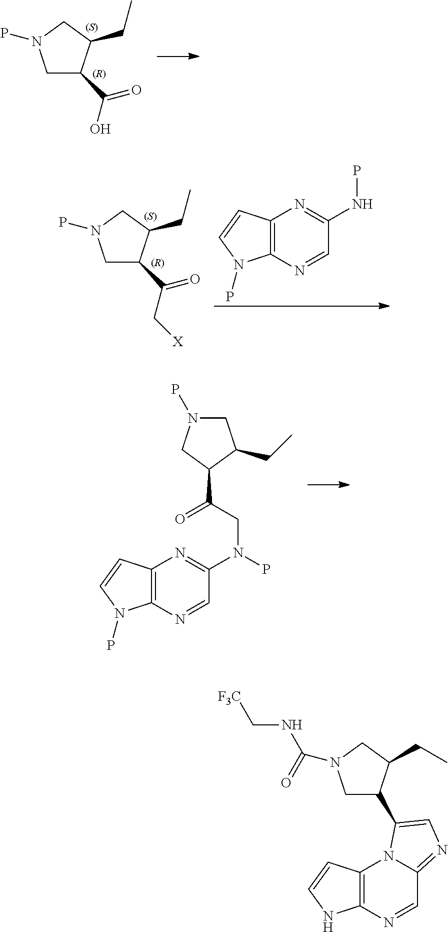

(3R,4S)-3-(2-haloacetyl)-4-ethylpyrrolidine-1-carboxylate may be converted to Upadacitinib according to any other methods known in the art or according to the procedures described or exemplified in the instant application.

Starting materials used in any aspect of the instant application may be obtained from either commercially available sources or prepared according to the methods known in the art. Starting materials used in any aspect of the instant application may be purified according to the methods known in the art such as recrystallization, acid-base treatment, chromatography, fractional distillation, slurrying or the like, before using.

Upadacitinib intermediate, pyrrolidine-3-carboxylate of formula (1) obtained according to any aspects of the instant patent application may be purified according to any of the methods known in the art recrystallization, acid-base treatment, chromatography or the like. Further, it may be dried under suitable drying conditions such as air drying or vacuum drying.

In another aspect, the present application provides a pharmaceutical composition comprising Upadacitinib obtained according any of the previous aspects and at least one additional pharmaceutically acceptable excipient.

In another aspect, the present application provides pyrrolidine-3-carboxylate of formula (1) or salt thereof, wherein its chemical purity of may be more than 99% by HPLC or more than 99.5% by HPLC or more than 99.9% by HPLC.

In another aspect, the present application provides novel intermediates as depicted below.

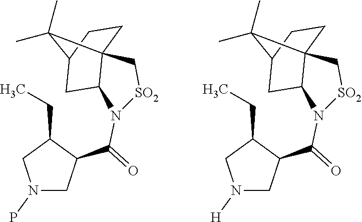

The present invention encompass processes for preparation of various derivatives of pyrrolidine-3-carboxylic acid of formula (1)

Certain specific aspects and embodiments of the present application will be explained in greater detail with reference to the following examples, which are provided only for purposes of illustration and should not be construed as limiting the scope of the application in any manner. Variations of the described procedures, as will be apparent to those skilled in the art, are intended to be within the scope of the present application.

### Definitions

The term “about” when used in the present application preceding a number and referring to it, is meant to designate any value which lies within the range of ±10%, preferably within a range of ±5%, more preferably within a range of ±2%, still more preferably within a range of ±1% of its value. For example “about 10” should be construed as meaning within the range of 9 to 11, preferably within the range of 9.5 to 10.5, more preferably within the range of 9.8 to 10.2, and still more preferably within the range of 9.9 to 10.1.

The term “inert solvent” when used in the present application is a solvent that does not react with the reactants or reagent s under conditions that cause the chemical reaction indicated to take place.

An “alcohol” is an organic compound containing a carbon bound to a hydroxyl group. “C1-C6 alcohols” include, but are not limited to, methanol, ethanol, ethylene glycol, diethylene glycol, 1-propanol, 2-propanol (isopropyl alcohol), 2-methoxyethanol, 1-butanol, 2-butanol, i-butyl alcohol, t-butyl alcohol, 2-ethoxyethanol, 1-, 2-, or 3-pentanol, neo-pentyl alcohol, t-pentyl alcohol, diethylene glycol monomethyl ether, diethylene glycol monoethyl ether, cyclohexanol, phenol, glycerol, or the like.

An “hydrocarbon” may be aliphatic or aromatic. An “aliphatic hydrocarbon” is a liquid hydrocarbon compound, which may be linear, branched, or cyclic and may be saturated or have as many as two double bonds. A liquid hydrocarbon compound that contains a six-carbon group having three double bonds in a ring is called “aromatic.” Examples of “C5-C8 aliphatic or aromatic hydrocarbons” include, but are not limited to, n-pentane, isopentane, neopentane, n-hexane, isohexane, 3-methylpentane, 2,3-dimethylbutane, neohexane, n-heptane, isoheptane, 3-methylhexane, neoheptane, 2,3-dimethylpentane, 2,4-dimethylpentane, 3,3-dimethylpentane, 3-ethylpentane, 2,2,3-trimethylbutane, n-octane, isooctane, 3-methylheptane, neooctane, cyclohexane, methylcyclohexane, cycloheptane, benzene, toluene, ethylbenzene, m-xylene, o-xylene, p-xylene, trimethylbenzene, chlorobenzene, fluorobenzene, trifluorotoluene, anisole, or any mixtures thereof.

An “ester” is an organic compound containing a carboxyl group —(C═O)—O— bonded to two other carbon atoms. “C3-C6esters” include, but are not limited to, ethyl acetate, n-propyl acetate, n-butyl acetate, isobutyl acetate, t-butyl acetate, ethyl formate, methyl acetate, methyl propanoate, ethyl propanoate, methyl butanoate, ethyl butanoate, or the like.

An “ether” is an organic compound containing an oxygen atom —O— bonded to two other carbon atoms. “C2-C6 ethers” include, but are not limited to, diethyl ether, diisopropyl ether, methyl t-butyl ether, glyme, diglyme, tetrahydrofuran, 2-methyltetrahydrofuran, 1,4-dioxane, dibutyl ether, dimethylfuran, 2-methoxyethanol, 2-ethoxyethanol, anisole, or the like.

A “halogenated hydrocarbon” is an organic compound containing a carbon bound to a halogen. Halogenated hydrocarbons include, but are not limited to, dichloromethane, 1,2-dichloroethane, trichloroethylene, perchloroethylene, 1,1,1-trichloroethane, 1,1,2-trichloroethane, chloroform, carbon tetrachloride, or the like.

A “ketone” is an organic compound containing a carbonyl group —(C═O)— bonded to two other carbon atoms. “C3-C6 ketones” include, but are not limited to, acetone, ethyl methyl ketone, diethyl ketone, methyl isobutyl ketone, ketones, or the like.

A “nitrile” is an organic compound containing a cyano —(C≡N) bonded to another carbon atom. “C2-C6Nitriles” include, but are not limited to, acetonitrile, propionitrile, butanenitrile, or the like.

### EXAMPLES

### Example-1: Preparation of diethyl 2-propylidenemalonate

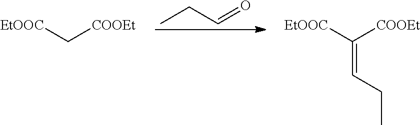

To mixture of diethyl malonate (50 g) and toluene (150 mL) at 27° C., piperidine (1.25 mL) and acetic acid (3.75 mL) was added and the stirred the reaction mixture for 15 minutes at the same temperature. Propionaldehyde (23.5 g) was added to the reaction mixture and heated to reflux temperature. Stirred the reaction mixture at reflux for 16 hours and quenched the reaction mixture with cold water (150 mL). Extracted the reaction mixture with diethyl ether (2×150 mL) and the combined organic layer was washed with 1 N Hydrochloric acid (150 mL), saturated sodium bicarbonate (150 mL) and brine solution (150 mL). The organic layer was dried on anhydrous sodium sulphate and evaporated to remove the solvent completely under reduced pressure at 45° C. The crude compound was purified by silica gel column chromatography (60-120 mesh) and 3% ethyl acetate-hexane as eluent to obtain the title compound. Yield: 20 g and Purity by HPLC: 87.56%

### Example-2: Preparation of diethyl 2-(1-nitrobutan-2-yl)malonate

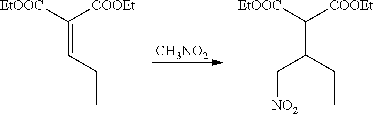

A mixture of diethyl 2-propylidenemalonate (20 g) and toluene (200 mL) was cooled to 15° C. and 1,8-diazabicyclo[5.4.0]undec-7-ene [DBU] (1.52 g) and nitromethane (7.32 g) were added slowly at the same temperature. Stirred the reaction mixture for 16 hours at 15° C. and evaporated the reaction mixture at 50° C. to remove the solvent. The crude product was purified by silica gel column chromatography (60-120 mesh) and 3% ethyl acetate-hexane as eluent to obtain the title compound. Yield: 11.0 g; Purity by HPLC: 94.66%

### Example-3: Preparation of ethyl 4-ethyl-2-oxopyrrolidine-3-carboxylate

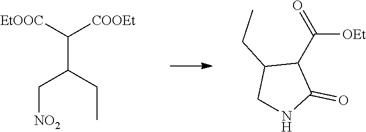 [SMILES: CCOC(=O)C(C(=O)OCC)C(CC)C[N+](=O)[O-] ; CCOC(=O)C1C(=O)NCC1CC]

To a mixture of diethyl 2-(1-nitrobutan-2-yl) malonate (11 g) in methanol (110 mL) at 27° C., 10% Pd-carbon (2.2 g) was added and degassed the reaction mixture with hydrogen pressure (60 psi). Stirred the reaction mixture for 16 hours at the same temperature pressure and filtered the reaction mixture on celite bed. The filtrate was evaporated under reduced at 50° C. and crude product was purified using silica gel column (60-120 mesh) and 4% methanol-dichloromethane as eluent to obtain the title compound. Yield: 6.0 g

### Example-4: Preparation of 1-(tert-butyl) 3-ethyl 4-ethyl-2-oxopyrrolidine-1,3-dicarboxylate

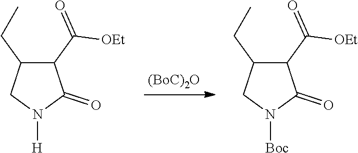 [SMILES: CCOC(=O)C1C(=O)NCC1CC ; CCOC(=O)C1C(=O)N(C(=O)OC(C)(C)C)CC1CC]

A mixture of Ethyl 4-ethyl-2-oxopyrrolidine-3-carboxylate (6.0 g) in dichloromethane (60 mL) was cooled to 0° C. and triethyl amine (3.27 g) was added slowly at the same temperature. (4-dimethylamino)pyridine (0.39 g) and then Di-tert-butyl dicarbonate (7.07 g) were added to the reaction mixture at 0° C. The reaction mixture was allowed to warm to 27° C. and stirred for 3 hours at the same temperature. Reaction mixture was quenched by adding to ice cold water (60 mL) and extracted with dichloromethane (2×60 mL). Washed the combined organic layer with water (60 mL) then with brine (60 mL) and dried over anhydrous sodium sulfate. The organic layer was evaporated completely and purified using silica gel column (60-120 mesh) and 10% ethyl acetate-hexane as eluent to obtain the title compound. Yield: 4.0 g and Purity by HPLC: 94.45%

### Example-5: Preparation of 1-(tert-butyl) 3-ethyl 4-ethyl-4,5-dihydro-1H-pyrrole-1,3-dicarboxylate

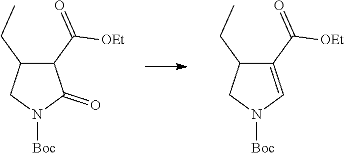 [SMILES: CCOC(=O)C1C(=O)N(C(=O)OC(C)(C)C)CC1CC ; CCOC(=O)C1=CN(C(=O)OC(C)(C)C)CC1CC]

A mixture of 1-(tert-butyl) 3-ethyl 4-ethyl-2-oxopyrrolidine-1,3-dicarboxylate (1.0 g) in methanol (10 mL) and toluene (10 mL) was cooled to −35° C. and sodium borohydride (0.397 g) was added. The reaction mixture was stirred at the same temperature for 1 hour and allowed to warm to −20° C. The reaction mixture was stirred at −20° C. for 2 hours and cooled to −40° C. The reaction mixture was quenched with 10% ammonium chloride solution and then extracted with toluene (2×10 mL). The organic layer was dried over anhydrous sodium sulfate and evaporated completely. The crude product was dissolved in toluene (10 mL) at 27° C. and Tulsion T-66 resin (100 mg) was added heated the mixture to 120° C. and stirred for 7 hours at the same temperature. Cooled the mixture and filtered through celite bed and washed the bed with toluene (10 mL). The filtrate was evaporated under reduced pressure and the crude product was purified using silica gel column (60-120 mesh) and 4% ethyl acetate-hexane as eluent to obtain the title compound. Yield: 456 mg and Purity by HPLC: 98.57%

### Example-6: Preparation of 1-(tert-butyl) 3-ethyl 4-ethylpyrrolidine-1,3-dicarboxylate

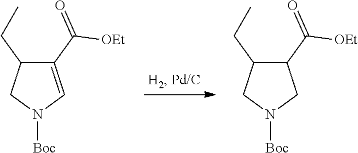 [SMILES: CCOC(=O)C1=CN(C(=O)OC(C)(C)C)CC1CC ; CCOC(=O)C1CN(C(=O)OC(C)(C)C)CC1CC]

To a mixture of 1-(tert-butyl) 3-ethyl 4-ethyl-4,5-dihydro-1H-pyrrole-1,3-dicarboxylate (0.15 g) in methanol (15 mL) at 27° C., 10% Pd-carbon (15 mg) was added and applied hydrogen pressure. Heated the reaction mixture to 40° C. and stirred at the same temperature for 4 hours. The reaction mixture was cooled to 27° C. and filtered through celite bed. Washed the bed with methanol and the filtrate was evaporated under reduced pressure. The crude product was purified by using silica gel column (100-200 mesh) and 2% ethyl acetate-hexane as eluent to obtain the title compound. Yield: 100 mg

### Example-7: Preparation of Hydrochloride salt of 4-ethylpyrrolidine-3-carboxylic acid

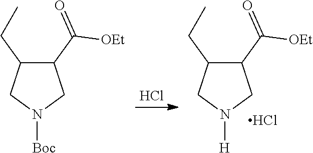 [SMILES: CCOC(=O)C1CN(C(=O)OC(C)(C)C)CC1CC ; Cl ; CCOC(=O)C1CNCC1CC ; Cl]

A mixture of 1-(tert-butyl) 3-ethyl 4-ethylpyrrolidine-1,3-dicarboxylate (100 mg) and 6 N hydrochloric acid (2 mL) was heated for 4 hours at 80° C. The reaction mixture was cooled to 27° C. and toluene (10 mL) was added. The reaction mixture was evaporated under reduced pressure to obtain the title compound. Yield: 60 mg

### Example-8: Preparation of 1-((benzyloxy)carbonyl)-4-ethylpyrrolidine-3-carboxylic acid

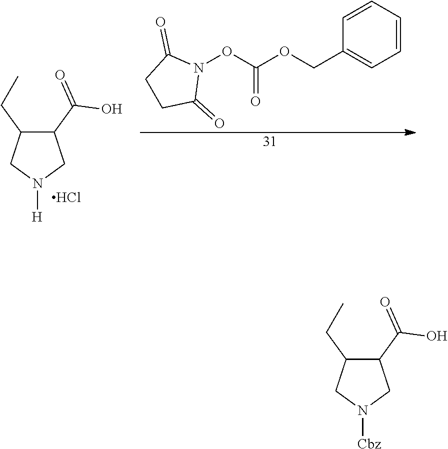

A mixture of Hydrochloride salt of 4-ethylpyrrolidine-3-carboxylic acid (60 mg), 1,4-dioxane (1.2 mL) and water (1.2 mL) was cooled to 0° C. and sodium carbonate (0.07 g) was added at the same temperature. -(Benzyloxycarbonyloxy)succinimide (0.12 g) was added to the reaction mixture and allowed to warm to 27° C. and stirred the reaction mixture for 16 hours at the same temperature. The reaction mixture was quenched with water (10 mL) and washed with ethyl acetate (2×10 mL). The aqueous layer was cooled to 0° C. and adjusted its pH to 2 using 1 N hydrochloric acid. The reaction mixture was extracted with (2×10 mL) and the combined organic layer was dried over anhydrous sodium sulfate. The organic layer was evaporated completely under reduced pressure and the crude product was purified by using silica gel column (100-200 mesh) and 2% methanol-dichloromethane as eluent to obtain the title compound. Yield: 50 mg and Purity by HPLC: 96.56%

### Example-9: Preparation of ethyl-2-pentenoate

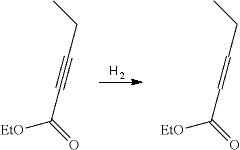

Taken Ethyl-2-pentynoate (100 g) and Lindlar catalyst (16 g) in Hexane (700 mL) and hydrogenated under 30 psi hydrogen pressure for 2 hours at 30° C. The reaction mixture was filtered and washed with hexane (3×100 mL). The filtrated was concentrated under reduced pressure at 35° C. to obtain the title compound as yellowish residue. Yield: 90.2 g

### Example-10: Preparation of racemic ethyl-1-benzyl-4-ethylpyrrolidine-3-carboxylate

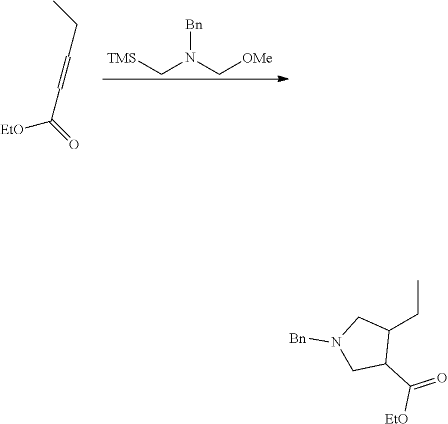

A mixture of Ethyl-2-pentenoate (90 g), N-benzyl-1-methoxy-N-trimethylsilyl methyl methanamine (165 g) and dichloromethane (630 mL) was cooled to −10° C. and trifluoroacetic acid (TFA) (0.54 mL) was added. Stirred the reaction mixture for 5 hours at −10° C. and allowed to warm to 30° C. The reaction mixture was stirred further for 48 hours at 30° C. and cooled to 15° C. Saturated sodium bicarbonate solution (1000 mL) was added to reaction mixture and stirred for 10 minutes at 15° C. Separated the organic layer and extracted the aqueous layer with dichloromethane (200 mL). The combined organic layer was washed with water (500 mL) and dried over sodium sulfate. Removed the solvent from the organic layer under reduced pressure at 40° C. and the crude product was purified by column chromatography using 10% ethyl acetate-hexane as eluent to obtain the title compound as yellow residue. Yield: 135.1 g

### Example-11: Preparation of racemic ethyl-4-ethylpyrrolidine-3-carboxylate

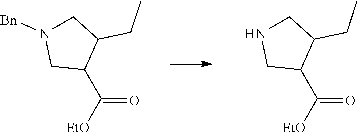 [SMILES: CCOC(=O)C1CN(Cc2ccccc2)CC1CC ; CCOC(=O)C1CNCC1CC]

A mixture of racemic ethyl-1-benzyl-4-ethylpyrrolidine-3-carboxylate (135 g) in methanol (810 mL) and palladium hydroxide on carbon (27 g) was hydrogenated with 60 psi pressure at 30° C. for 16 hours. The reaction mixture was filtered celite bed and the washed the bed with methanol (250 mL). The solvent was removed from the filtrate under reduced pressure at 50° C. to obtain the title compound as pale brown residue. Yield: 90.3 g

### Example-12: Preparation of ethyl 1-benzyl-4-ethyl-2,5-dihydro-1H-pyrrole-3-carboxylate

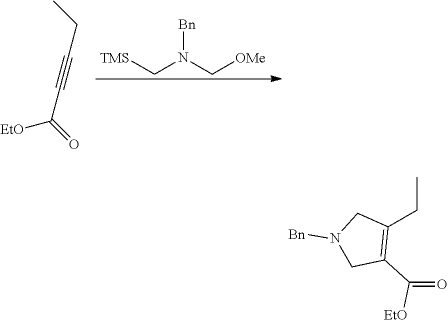

A mixture of ethyl-2-pentnoate (25 g), N-benzyl-1-methoxy-N-trimethylsilyl) methane amine (94.09 g) and dichloromethane (250 mL) was cooled to −5° C. and Trifluoro acetic acid (3.05 mL) was added to it slowly. The reaction mixture was stirred for 1 hour at −5° C. and for 2 hours at 0° C. The temperature of the reaction mixture was allowed to reach 27° C. and stirred at the same temperature for 16 hours. The solvent was removed under reduced pressure after the completion of reaction and purified the crude product by column chromatography using 4% ethyl acetate-hexane mixture as eluent to obtain the title compound as yellow liquid. Yield: 29.2 g

### Example-13: Preparation of ethyl 1-benzyl-4-ethylpyrrolidine-3-carboxylate

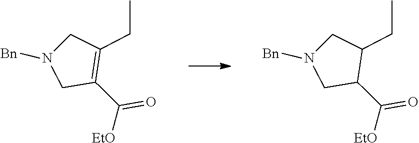 [SMILES: CCOC(=O)C1=C(CC)CN(Cc2ccccc2)C1 ; CCOC(=O)C1CN(Cc2ccccc2)CC1CC]

Ethyl 1-benzyl-4-ethyl-2,5-dihydro-1H-pyrrole-3-carboxylate (200 g) was dissolved in methanol (1400 mL) and Raney Ni (40 g, which was washed with water (3×200 mL) and methanol (3×200 mL) was added. The reaction mixture was stirred at 27° C. for 5 hours under hydrogen pressure (30 psi) and filtered through celite bed. The celite bed was washed with methanol (200 mL). The solvent was removed from the filtrate under reduced pressure at 45° C. and the crude product was purified by column chromatography using 15% ethyl acetate-hexane as eluent to obtain the title compound as yellow liquid. Yield: 110 g

### Example-14: Preparation of racemic 4-ethylpyrrolidine-3-carboxylic acid

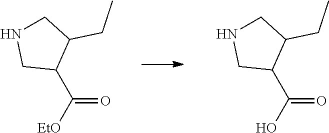

A mixture of racemic ethyl-4-ethylpyrrolidine-3-carboxylate (90 g) and 6N HCl (990 mL) was heated to 100° C. for 16 hours. The water and HCl were removed from the reaction mixture under reduced pressure at 55° C. to obtain the title compound as yellow solid. Yield: 75.2 g

### Example-15: Preparation of racemic 1-carbobenzyloxy-4-ethylpyrrolidine-3-carboxylic acid

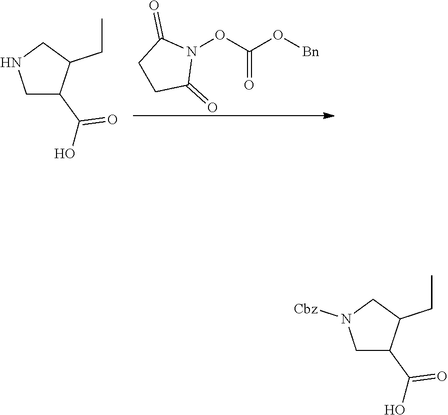

Racemic 4-ethylpyrrolidine-3-carboxylic acid (75 g) was dissolved in 1,4-dioxane (750 mL) at 28° C. and cooled the solution to 7° C. Sodium carbonate (77.8 g) was added to the solution to adjust the pH of the solution to 8. N-Benzyloxycarbonyloxy)succinimide (156.7 g) was added to the reaction mixture and warmed to 28° C. The reaction mixture was stirred for 16 hours at the same temperature and extracted with ethyl acetate (2×562 mL). The organic layer was washed with saturated sodium bicarbonate solution (2×500 mL). The aqueous layer was acidified with concentrated HCl to pH 4 and extracted with ethyl acetate (2×750 mL). The combined organic layer was washed with water (2×1000 mL), brine solution (500 mL) and dried over sodium sulfate. The solvent was removed under reduced pressure at 45° C. to obtain the title compound as pale yellow residue. Yield: 104.3 g

### Example-16: Resolution of 1-carbobenzyloxy-4-ethylpyrrolidine-3-carboxylic acid

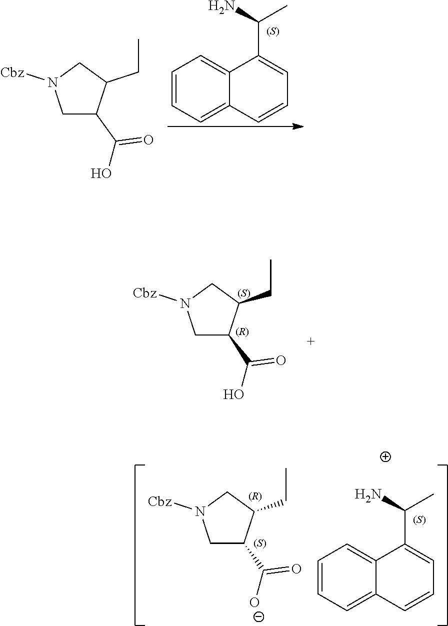

To a mixture of 1-carbobenzyloxy-4-ethylpyrrolidine-3-carboxylic acid (104 g) in acetonitrile (416 mL) at 30° C., (S)-1-(naphthalen-1-yl)ethan-1-amine (21 mL) was added and stirred for 16 hours at the same temperature. The solid was filtered and washed with acetonitrile (2×50 mL). The filtrate was concentrated and washed with 1N hydrochloride (2×150 mL). The aqueous layer was extracted with dichloromethane (500 mL). The combined organic layer was washed with water (2×200 mL), brine solution (200 mL) and dried over sodium sulfate. The solvent was removed from the organic layer under reduced pressure at 40° C. to obtain the title compound as yellow residue.

### Yield: 60.2 g; Chiral Purity: R-Isomer: 62.98% & S-isomer: 29.28%.

### Example-17: Preparation of (R)-1-(naphthalen-1-yl)ethan-1-amine salt of (3R,4S)-1-((benzyloxy)carbonyl)-4-ethylpyrrolidine-3-carboxylic acid

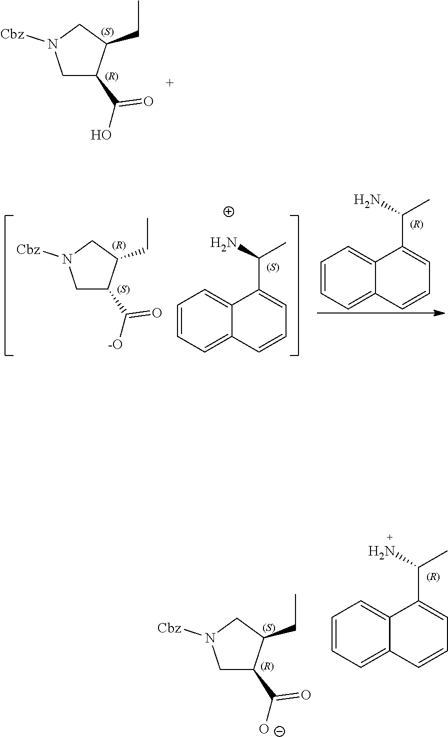

To a mixture of the resolution product (70 g) obtained in previous example and acetonitrile (280 mL), (R)-1-(naphthalen-1-yl) ethan-1-amine (22.04 mL) was added at 30° C. and the reaction mixture was stirred for 16 hours at the same temperature. The solid was filtered and the washed with acetonitrile (2×50 mL). The solid was dried under reduced pressure at 30° C. for 2 hours to obtain the title compound as white solid.

Yield: 41.7 g; Chemical Purity by HPLC: 97.50; and chiral purity by HPLC: R-isomer: 98.69%

### Example-18: Preparation of benzyl (3R,4S)-3-(2-bromoacetyl)-4-ethylpyrrolidine-1-carboxylate

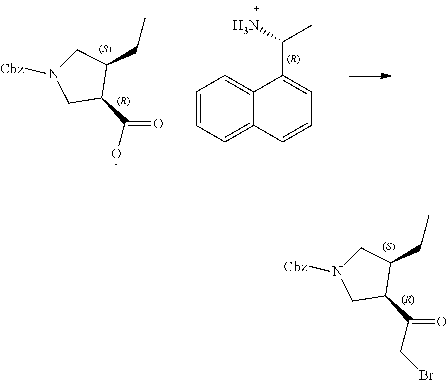

1N HCl (1090 mL) & dichloromethane (500 mL) were added to (R)-1-(naphthalen-1-yl) ethan-1-amine salt of (3R,4S)-1-((benzyloxy)carbonyl)-4-ethylpyrrolidine-3-carboxylic acid (109 g) and stirred. The organic layer was separated and aqueous layer was extracted with dichloromethane (1090 mL). Combined organic layer was washed with 1N HCl (1090 mL), brine solution (1090 mL) and dried over sodium sulfate. The solvent was removed under reduced pressure at 40° C. to obtain crude product. The crude product was dissolved in dichloromethane (654 mL) and cooled to 5° C. 2M oxalyl chloride in dichloromethane (243 mL) was added at 5° C. and stirred for 10 min at the same temperature. Dimethyl formamide (0.2 mL) was added at 5° C. and stirred at 27° C. for 6 hours. The solvent was removed under reduced pressure at 45° C. and diluted the reaction mixture with tetrahydrofuran and acetonitrile and cooled to −10° C. under nitrogen atmosphere. 2M Trimethylsilyldiazomethane in ether (486.6 mL) was added at −10° C. and stirred for 3 hours at the same temperature. 47% aqueous HBr was added at −5° C. and stirred for 3 hours. After the completion of reaction, water (1090 mL) and methyl tert.butyl ether (1090 mL) was added at 0° C. and separated the organic layer. The organic layer was washed with saturated sodium bicarbonate solution, brine solution and dried over sodium sulfate. The solvent was removed from the organic layer under reduced pressure and purified the crude product by column chromatography using 15% ethyl acetate-hexane as eluent to obtain the title compound as orange solid. Yield: 70 g

### Example-19: Preparation of pent-2-ynoic acid

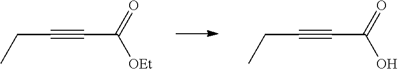

To a mixture of ethyl pent-2-ynoate (10 g) and tetrahydrofuran (25 mL) at 27° C., aqueous solution of lithium hydroxide monohydrate (6.54 g in 90 mL of water) was added and stirred for 90 minutes at the same temperature. The pH of the reaction mixture adjusted to 2 at 0° C. with concentrated HCl. The reaction mixture was extracted with methyl tert-butyl ether (500 mL) and the organic layer was dried over sodium sulfate. The solvent was removed from the organic layer under reduced pressure at 35° C. to obtain the title compound. Yield: 6.89 g, purity by HPLC: 99.70%

### Example-20: Preparation of (1R)-(+)-N-(pent-2-ynoyl)-2,10-Camphorsultam

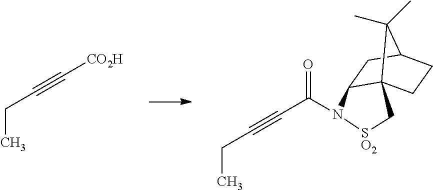

A solution of Pent-2-ynoic acid (2.28 g) in tetrahydrofuran (100 mL) was cooled to −78° C. and Pivaloyl chloride (3.03 mL) and triethylamine (3.43 mL) were added slowly to the above solution at the same temperature. Stirred the reaction mixture, at −78° C. for 15 minutes and then at 0° C. for 30 minutes. Again cooled the reaction mixture to −78° C. to obtain a solution of the pivaloyl pent-2-ynoyl mixed anhydride. n-Butyl lithium (9.76 mL) was added to a mixture of (1R)-(+)-2, 10-camphorsultam (5.0 g) and anhydrous THF (50 mL) at −78° C. and stirred this mixture for 15 minutes at the same temperature. This mixture was added to the above mixed anhydride solution at −78° C. and stirred for 45 minutes. The temperature of the reaction mixture was increased to 30° C. and 5% aqueous citric acid solution (100 mL) was added into the reaction mixture. The reaction mixture was extracted with ethyl acetate (2×100 mL) and washed the organic layer with saturated sodium bicarbonate solution (100 mL), brine solution (100 mL) and dried over anhydrous sodium sulfate. The solvent was removed from the organic layer under reduced pressure to obtain the crude product which was purified by column chromatography using 20% ethyl acetate-hexane as eluent on 60-120 mesh silica gel to obtain the title compound as white get solid. Yield: 5.08 g, purity by HPLC: 98.96%

### Example-21: Preparation of (1R)-(+)-N-(pent-2-enoyl)-2,10-Camphorsultam

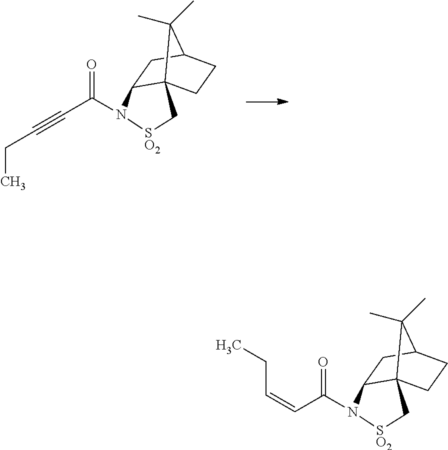 [SMILES: CCC#CC(=O)N1[C@H]2CC3CC[C@@]2(CS1(=O)=O)C3(C)C ; CC/C=C\C(=O)N1[C@H]2CC3CC[C@@]2(CS1(=O)=O)C3(C)C]

To a mixture of (1R)-(+)-N-(pent-2-ynoyl)-2,10-Camphorsultam (1 g), ethyl acetate (10 mL) and tetrahydrofuran (10 mL), Lindlar's catalyst (200 mg) was added at 30° C. and stirred under hydrogen atmosphere for 6 hours at the same temperature. The reaction mixture was filtered through celite bed and washed the bed with ethyl acetate (2×25 mL). The solvent was removed from the filtrate under reduced pressure to obtain the title compound as pale yellow oil. Yield: 1.0 g, purity by HPLC: 97.52%

### Example-22: Preparation of (1R)-(+)-N-((3R,4S)-1-benzyl-4-ethylpyrrolidine-3-carbonyl)-2,10-Camphorsultam

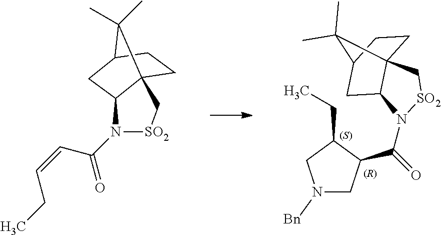

A mixture of (1R)-(+)-N-(pent-2-enoyl)-2,10-Camphorsultam (1.0 g) and dichloromethane (10 mL) was cooled to 0° C. and N-Benzyl-N-(methoxymethyl)-N-trimethylsilylmethylamine (1.30 mL) and trifluoroacetic acid (77 mg in 1.0 mL dichloromethane) were added. Stirred the reaction mixture at 0° C. for 48 h and the solvent was removed from the reaction mixture under reduced pressure at 30° C. The product was purified by column chromatography using 20% ethyl acetate-hexane as eluent on 60-120 mesh silica gel to obtain the title compound as a white solid. Yield: 1.14 g, purity by HPLC: 96.16%

### Example-23: Preparation of (3R,4S)-1-benzyl-4-ethylpyrrolidine-3-carboxylic acid

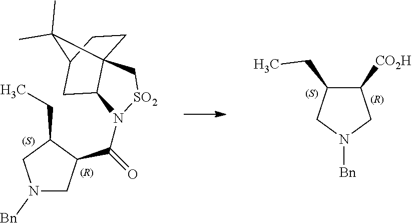

A mixture of (1R)-(+)-N-((3R,4S)-1-benzyl-4-ethylpyrrolidine-3-carbonyl)-2,10-Camphorsultam (3.3 g) and tetrahydrofuran (30 mL) was cooled to 0° C. and a solution of Lithium hydroxide (1.61 g) monohydrate dissolved in water (30 mL) was added into the reaction mixture at 0° C. The reaction mixture was stirred for 24 hours at 30° C. The solvent was removed from the reaction mixture under reduced pressure and water (30 mL) was added to the reaction mixture. The reaction mixture was washed with ethyl acetate (100 mL) and adjusted the pH to 3 using 3M HCl. Sodium chloride was added to the reaction mixture and extracted with ethyl acetate (100 mL). The organic layer was dried over sodium sulfate and the solvent was removed under reduced pressure to obtain the title compound as white solid. Yield: 1.6 g, purity by HPLC: 93.98%, Chiral purity by HPLC: 95.9%.

### Example-24: Preparation of benzyl (3R,4S)-3-(N-(tert-butoxycarbonyl)-N-(5-tosyl-5H-pyrrolo[2,3-b]pyrazin-2-yl)glycyl)-4-ethylpyrrolidine-1-carboxylate

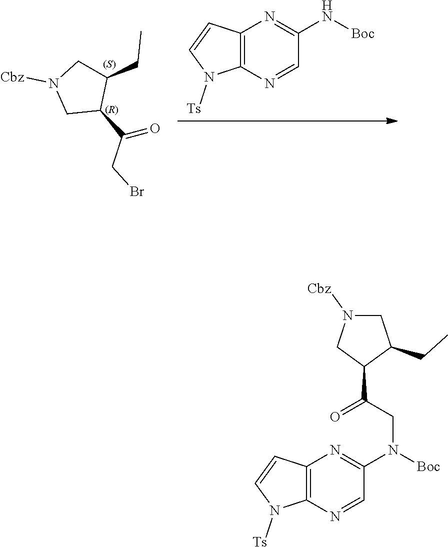

A mixture of Sodium hydride (6.88 g) (60% dispersion in oil) and dimethyl formamide (305 mL) were cooled to 0° C. A solution of tert-butyl (5-tosyl-5H-pyrrolo[2,3-b]pyrazin-2-yl)carbamate (66.89 g) in dimethylformamide (305 g) was added to the above mixture at 0° C. and stirred for 90 minutes at the same temperature. A mixture of Benzyl (3R,4S)-3-(2-bromoacetyl)-4-ethylpyrrolidine-1-carboxylate (61 g) in dimethylformamide (305 mL) at 0° C. was added to above reaction mixture and stirred at the same temperature for 1 hour. After the completion of the reaction, cold water (3.0 L) was added to the reaction mixture and extracted with methyl tert.butyl ether (3×700 mL). The combined organic layer was washed with water (2×800 mL), brine solution (800 mL) and dried over sodium sulfate. The solvent was removed from the organic layer under reduced pressure at 42° C. and the crude product was purified by column chromatography 100-200 mesh using 35% ethyl acetate-hexane as eluent to obtain the title compound as an off-white solid. Yield: 69.7 g; Chemical purity by HPLC: 96.36%; and Chiral purity by HPLC: 99.11%

### Example-25: Preparation of benzyl (3S,4R)-3-ethyl-4-((5-tosyl-5H-pyrrolo[2,3-b]pyrazin-2-yl)glycyl)pyrrolidine-1-carboxylate

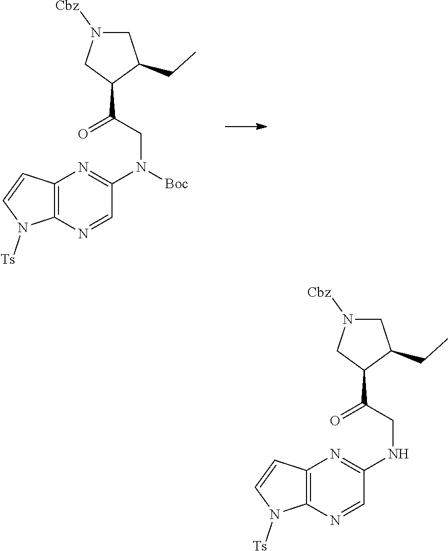 [SMILES: CC[C@@H]1CN(C(=O)OCc2ccccc2)C[C@@H]1C(=O)CN(C(=O)OC(C)(C)C)c1cnc2c(ccn2S(=O)(=O)c2ccc(C)cc2)n1 ; CC[C@@H]1CN(C(=O)OCc2ccccc2)C[C@@H]1C(=O)CNc1cnc2c(ccn2S(=O)(=O)c2ccc(C)cc2)n1]

A mixture of Benzyl (3R,4S)-3-(N-(tert-butoxycarbonyl)-N-(5-tosyl-5H-pyrrolo[2,3-b]pyrazin-2-yl)glycyl)-4-ethylpyrrolidine-1-carboxylate (53 g) and dichloromethane (636 mL) was cooled 5° C. and trifluoroacetic acid (37.2 mL) was added. Allowed the reaction mixture to warm to 27° C. and stirred for 4 hours at the same temperature. The reaction mixture was added to cold saturated sodium bicarbonate solution (1.0 L) and the organic layer was separated. Aqueous layer was extracted with dichloromethane (530 mL) and separated the organic layer. The combined organic layer was washed with brine solution (1 L) and dried over sodium sulfate. The solvent was removed from the organic layer under reduced pressure at 42° C. to obtain the title compound as pale brown solid. Yield: 42.4 g; Chemical purity by HPLC: 98.17%; and Chiral purity by HPLC: 99.69%

### Example-26: Preparation of benzyl (3S,4S)-3-ethyl-4-(3-tosyl-3H-imidazo[1,2-a]pyrrolo[2,3-e]pyrazin-8-yl)pyrrolidine-1-carboxylate

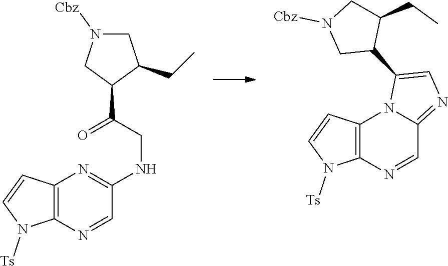

A mixture of benzyl (3S,4R)-3-ethyl-4-((5-tosyl-5H-pyrrolo[2,3-b]pyrazin-2-yl)glycyl)pyrrolidine-1-carboxylate (42 g), 1,4-dioxane (210 mL) and toluene (840 mL) was degassed with argon gas for 40 minutes at 27° C. Lawesson's reagent (22.68 g) was added to the reaction mixture at 27° C. and heated to 80° C. Stirred the reaction mixture for 4 hours at the same temperature and cooled to back to 27° C. after the completion of reaction. Water (210 mL) and Sodium perborate monohydrate (37.3 g) were added to the reaction mixture and stirred for 16 hours at 27° C. Saturated sodium bicarbonate solution (210 mL) was added to the reaction mixture and stirred for 2 hours at 27° C. Separated the organic layer and the aqueous layer was extracted with ethyl acetate (2×210 mL). The combined organic layer was washed with brine solution (420 mL) and dried the organic layer over sodium sulfate. The solvent was removed from the organic layer under reduced pressure at 45° C. and purified the crude product by column 100-200 mesh chromatography using 65% ethyl acetate-hexane mixture to obtain title compound as pale yellow solid. Yield: 34.8 g

### Example-27: 8-((3S,4S)-4-ethylpyrrolidin-3-yl)-3-tosyl-3H-imidazo[1,2-a]pyrrolo[2,3-e]pyrazine

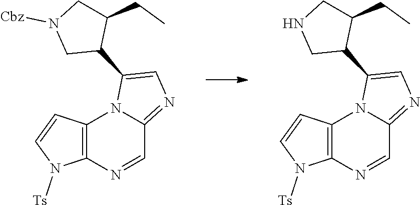

To a solution of benzyl (3S,4S)-3-ethyl-4-(3-tosyl-3H-imidazo[1,2-a]pyrrolo[2,3-e]pyrazin-8-yl)pyrrolidine-1-carboxylate (54.5 g) in 1,4-dioxane (1090 mL), 33% HBr in acetic acid (123 mL) was added slowly at 27° C. and heated to 60° C. The reaction mixture was stirred at the same temperature for 3 hours and cooled to 27° C. Water (2.2 L) and ethyl acetate (500 mL) was added to the reaction mixture and separated the organic layer and discarded. The aqueous layer was washed with ethyl acetate (2×500 mL). The pH of the aqueous layers was adjusted to 7.5 using sodium bicarbonate and then extracted with 10% 2-propanol-dichloromethane (3×500 mL). The organic layer was washed with brine solution (500 mL) and dried over sodium sulfate. The solvent was removed from the organic layer under reduced pressure at 42° C. to obtain pale brown solid. Yield: 41.5 g and Purity by HPLC: 86.13%

### Example-28: (3S,4S)-3-ethyl-4-(3-tosyl-3H-imidazo[1,2-a]pyrrolo[2,3-e]pyrazin-8-yl)-N-(2,2,2-trifluoroethyl)pyrrolidine-1-carboxamide

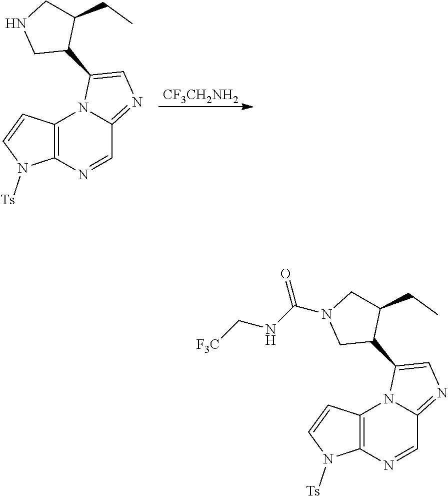

To a mixture of 1,1-carbonyl diimidazole (40.58 g) and dimethylformamide (82.0 mL) at 27° C., 2,2,2-trifluoro ethylamine (24.8 g) was added and heated the reaction mixture to 65° C. The reaction mixture was stirred for 16 hours at the same temperature for 16 hours and cooled to 27° C. The reaction mixture was added to a mixture of 8-((3S,4S)-4-ethylpyrrolidin-3-yl)-3-tosyl-3H-imidazo[1,2-a]pyrrolo[2,3-e]pyrazine (41.0 g) and dimethylformamide (328 mL) at 27° C. and heated to 65° C. The reaction mixture was stirred for 1 hour at the same temperature and cooled to 27° C. after the completion of reaction. The reaction mixture was added to cold water (1230 mL) and extracted with 10% Isopropyl alcohol-dichloromethane mixture (3×300 mL). The combined organic layer was washed with cold water (2×400 mL) and brine solution (400 mL). The organic layer was dried over sodium sulphate and removed the solvent under reduced pressure at 42° C. The crude product was purified by 60-120 mesh column chromatography using 5% methanol-dichloromethane mixture as eluent to obtain pale brown residue. Yield: 49.52 g and Purity by LC-MS: 85.97%

### Example-29: Preparation of Upadacitinib

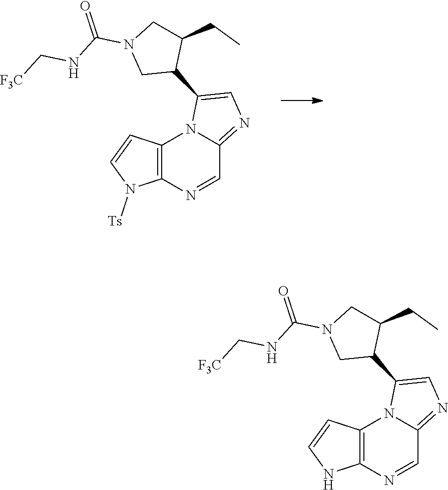

To a solution of (3S,4S)-3-ethyl-4-(3-tosyl-3H-imidazo[1,2-a]pyrrolo[2,3-e]pyrazin-8-yl)-N-(2,2,2-trifluoroethyl)pyrrolidine-1-carboxamide (11.2 g) in 1,4-dioxane (168 mL), 1N sodium hydroxide (2.52 g of sodium hydroxide dissolved in 63 mL of water) was added at 27° C. and heated to 55° C. The reaction mixture was stirred at same temperature for 1 hour and cooled to 27° C. after the completion of reaction. Water (200 mL) was added to the reaction mixture and extracted with ethyl acetate (4×140 mL). The combined organic layer was washed with brine solution (140 mL) and dried over sodium sulphate. The solvent was removed under reduced pressure at 42° C. The crude product was purified by column chromatography using 8% methanol-dichloromethane mixture as eluent to obtain pale brown solid. The solid was suspended in a mixture of heptane (25 mL) and acetone (5 mL) for 30 minutes at 27° C. and the solid was filtered. The solid was dried under reduced pressure for 30 minutes at 50° C. to obtain the title compound as pale brown solid. Yield: 5.52 g; chemical purity by HPLC: 99.69% and chiral purity by HPLC: 99.73%.

1. A process for the preparation of Upadacitinib intermediate, pyrrolidine-3-carboxylic acid, its ester or a salt thereof of formula (1), the process comprising the steps of:
a) cyclizing 2-(1-nitrobutan-2-yl)malonate of formula (c) to 2-oxopyrrolidine-3-carboxylate of formula (d); 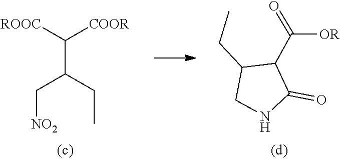
b) optionally, protecting 2-oxopyrrolidine-3-carboxylate of formula (d);
c) reducing the 2-oxopyrrolidine-3-carboxylate of formula (d) to pyrrolidine-3-carboxylic acid or an ester thereof of formula (1); 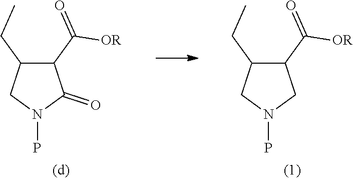
d) optionally, removing the protecting group of step b);
wherein R is hydrogen or a group selected from the group comprising of alkyl, aryl, arylalkyl; and P is a hydrogen (or) nitrogen protecting group selected from the group comprising of tert-Butyloxycarbonyl (Boc), Benzyloxycarbonyl (Cbz), Benzyl (Bn).

2. The process of claim 1, wherein the cyclization of 2-(1-nitrobutan-2-yl) malonate of formula (c) is carried out by hydrogenation.

3. The process of claim 1, wherein the cyclization of 2-(1-nitrobutan-2-yl) malonate of formula (c) is carried out by hydrogenating with molecular hydrogen in the presence of a catalyst.

4. The process of claim 3, wherein the catalyst is selected from the group comprising of palladium-on-carbon, platinum (IV) oxide and Raney™ nickel.

5. The process of claim 1, wherein reducing 2-oxopyrrolidine-3-carboxylate of formula (d) is carried out through the formation of 4,5-dihydro-1H-pyrrole-3-carboxylate of formula (d-i) and its hydrogenation.

6. The process of claim 1, wherein reducing 2-oxopyrrolidine-3-carboxylate of formula (d) is carried out without isolating the intermediate, 4,5-dihydro-1H-pyrrole-3-carboxylate of formula (d-i).

7. The process of claim 1, wherein reducing 2-oxopyrrolidine-3-carboxylate of formula (d) is carried out in the presence of one or more reducing agents.

8. The process of claim 7, wherein the reducing agent is selected from the group comprising of hydrogenation agents: palladium-on-carbon, platinum (IV) oxide, or Raney™ nickel; hydrides of boron and Aluminium: sodium borohydride, potassium borohydride, Lithium borohydride, sodium cyano borohydride, lithium alkyl borohydride, sodium dihydro-bis-(2-methoxyethoxy) aluminate solution (VITRIDE®), diisobutyl aluminium hydride; or combinations thereof.

9. The process of claim 1, wherein cyclization of 2-(1-nitrobutan-2-yl) malonate of formula (c) is carried under metal mediated reduction conditions using agents selected from the group comprising of zinc and acetic acid, zinc and hydrochloric acid, tin and hydrochloric acid, sodium amalgam in ethanol, or iron and acetic acid; tin chloride (II), titanium (III) chloride or combinations thereof.

10. The process of claim 1, wherein 2-(1-nitrobutan-2-yl) malonate of formula (c) can be obtained by the process comprising the steps of:
a) reacting dialkyl malonate of formula (a) with Propionaldehyde to obtain dialkyl 2-propylidenemalonate of formula (b) 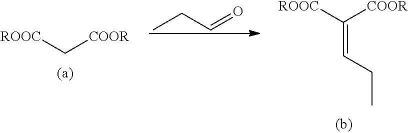
b) treating dialkyl 2-propylidenemalonate of formula (b) with nitromethane to obtain 2-(1-nitrobutan-2-yl) malonate of formula (c).

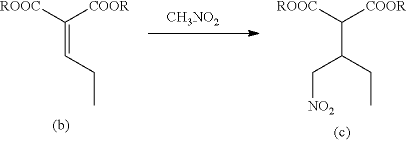

11. A process for the preparation of Upadacitinib intermediate, pyrrolidine-3-carboxylic acid, its derivative or a salt thereof of formula (1), comprising the step of cyclizing alkyl pent-2-enoate by treating it with an amine compound, 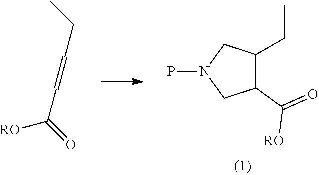
wherein R is hydrogen or group selected from the group comprising of alkyl, aryl, arylalkyl; and P is a nitrogen protecting group selected from the group comprising of tert-Butyloxycarbonyl (Boc), Benzyloxycarbonyl (Cbz), Benzyl (Bn).

12. A process for the preparation of Upadacitinib intermediate, pyrrolidine-3-carboxylic acid, its ester or a salt thereof of formula (1), the process comprising the steps of:
a) cyclizing the alkyl pent-2-ynoate to obtain alkyl 4-eth-2,5-dihydro-1H-pyrrole-3-carboxylate 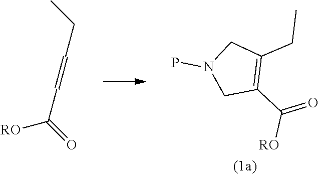
b) reducing the alkyl 4-ethyl-2,5-dihydro-1H-pyrrole-3-carboxylate of step a) to obtain compound of formula (1) 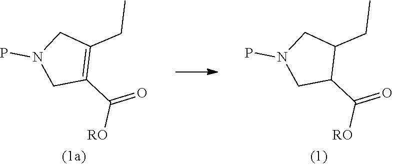
wherein R is hydrogen or group selected from the group comprising of alkyl, aryl, arylalkyl; and P is a nitrogen protecting group selected from the group comprising of tert-Butyloxycarbonyl (Boc), Benzyloxycarbonyl (Cbz), Benzyl (Bn).

13. A process for the preparation of Upadacitinib intermediate, pyrrolidine-3-carboxylic acid, its derivative or a salt thereof of formula (1), the process comprising the steps of:
a) reacting pent-2-ynoic acid or its derivative thereof with an optically active sultam compound to obtain pent-2-ynamide 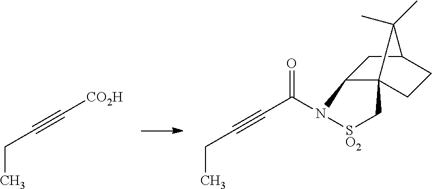
b) reducing the pent-2-ynamide of step a) to corresponding pent-2-enamide 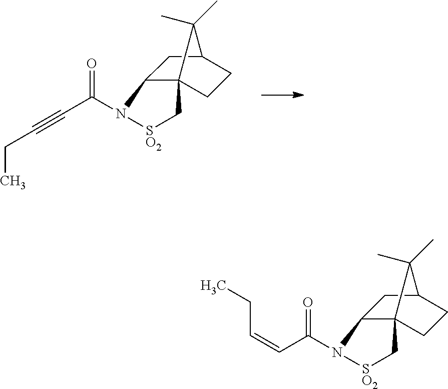 [SMILES: CCC#CC(=O)N1[C@H]2CC3CC[C@@]2(CS1(=O)=O)C3(C)C ; CC/C=C\C(=O)N1[C@H]2CC3CC[C@@]2(CS1(=O)=O)C3(C)C]
c) cyclizing the pent-2-enamide obtained in step b) to obtain pyrrolidine-3-carbamide, wherein P is hydrogen or a nitrogen protecting group 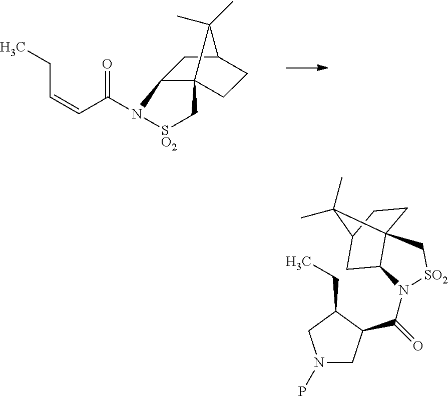
d) hydrolyzing the pyrrolidine-3-carbamide of step c) to obtain pyrrolidine-3-carboxylic acid

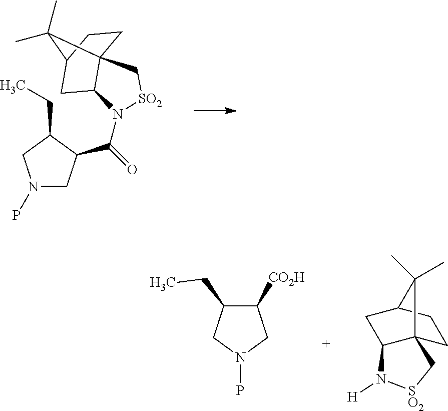

14. A process for the preparation of Upadacitinib intermediate, pyrrolidine-3-carboxylic acid, its ester or a salt thereof of formula (1), the process comprising the steps of:
a) cyclizing the pent-2-enamide to obtain pyrrolidine-3-carbamide, wherein P is hydrogen or a nitrogen protecting group 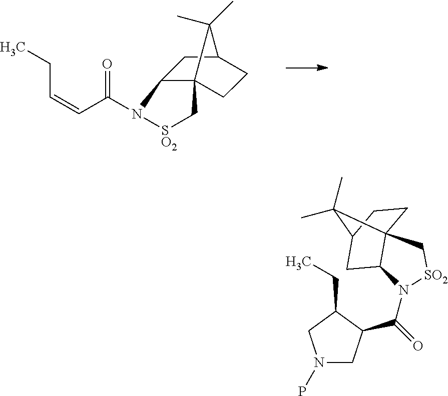
b) hydrolyzing the pyrrolidine-3-carbamide of step c) to obtain pyrrolidine-3-carboxylic acid

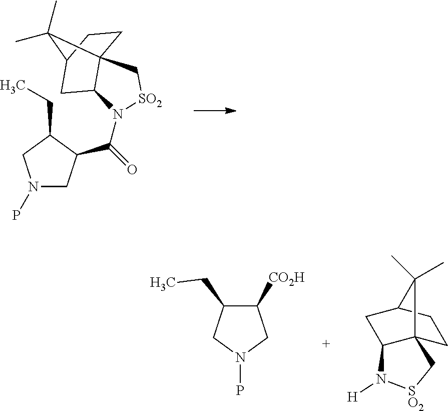

15. A process for the preparation of Upadacitinib or a salt thereof, the process comprising the steps of:
a) resolving pyrrolidine-3-carboxylic acid, its derivative or a salt thereof of formula (1) with an optically active compound, and
b) converting the optically active isomer of pyrrolidine-3-carboxylic acid, its derivative or a salt thereof obtained in step a) to Upadacitinib or a salt thereof.

16. The process of claim 15, wherein the optically active compound is selected from the group comprising of R-1-Naphthyl ethyl amine, S-1-Naphthyl ethyl amine, R-1-phenyl ethyl amine and S-1-phenyl ethyl amine.

17. A process for the preparation of Upadacitinib or a salt thereof, comprising the process for preparing pyrrolidine-3-carboxylate of formula (1) according to claim 1 and converting it to Upadacitinib or a salt thereof.

18. Pyrrolidine-3-carboxamide intermediates of following formula, wherein P is a nitrogen protecting group.

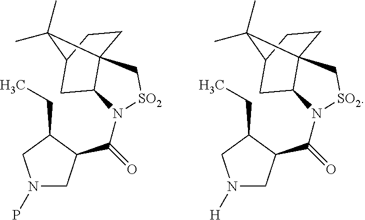

19. A process for the preparation of Upadacitinib or a salt thereof, comprising the process for preparing pyrrolidine-3-carboxylate of formula (1) according to claim 11 and converting it to Upadacitinib or a salt thereof.

20. A process for the preparation of Upadacitinib or a salt thereof, comprising the process for preparing pyrrolidine-3-carboxylate of formula (1) according to claim 12 and converting it to Upadacitinib or a salt thereof.

21. A process for the preparation of Upadacitinib or a salt thereof, comprising the process for preparing pyrrolidine-3-carboxylate of formula (1) according to claim 13 and converting it to Upadacitinib or a salt thereof.

22. A process for the preparation of Upadacitinib or a salt thereof, comprising the process for preparing pyrrolidine-3-carboxylate of formula (1) according to claim 14 and converting it to Upadacitinib or a salt thereof.
